(19) United States
(12) Patent Application Publication
(10) Pub. No.: US 2025/0090207 A1
(43) Pub. Date: Mar. 20, 2025

(54) TARGETED PLATE-NAIL CONSTRUCTS

(71) Applicant: GLOBUS MEDICAL, INC., AUDUBON, PA (US)

(72) Inventor: David Machamer, Glen Mills, PA (US)

(21) Appl. No.: 18/467,780

(22) Filed: Sep. 15, 2023

(51) Int. Cl. A61B 17/80; A61B 17/17; A61B 17/72

(52) U.S. Cl. CPC A61B 17/8061; A61B 17/1728; A61B 17/7241; A61B 17/808

(57) ABSTRACT

Devices, systems, instruments, and methods for promoting healing and stability for bone fractures. A targeted interlocking plate-nail system may include an intramedullary nail configured to extend through a medullary canal of a long bone, a bone plate configured to be positioned against an exterior surface of the long bone, and a plurality of fasteners configured to extend through the bone plate, through the intramedullary nail, or through both the bone plate and the intramedullary nail, thereby interlocking the bone plate and the intramedullary nail together. Targeting instruments may be used for guiding the components into position.

### FIELD OF THE INVENTION

The present disclosure relates to surgical devices, and more particularly, to stabilization systems, for example, for trauma applications.

### BACKGROUND OF THE INVENTION

Bone fractures of long bones, such as the femur, tibia, and humerus, may be treated by fixation of the bone to stabilize and align the fractured bone, facilitating the healing process. In some cases, one or more plates may be held against the fractured bone with screws, for example, to provide a compressive force. A single locking plate construct may be used to treat intra-articular fractures as well as extra-articular, shaft, and peri-prosthetic fractures. A single locking plate construct may provide for multiple distal/proximal fixation options and improved construct stiffness. Some disadvantages of locked plating, however, may include soft tissue disruption, less than ideal biomechanical loading, and delayed weight bearing.

In some cases, when a bone is fractured, an intramedullary nail may be introduced through the medullary cavity of the bone. A single intramedullary nail construct may be used to treat extra-articular, shaft, and peri-prosthetic fractures with the added benefits of minimal soft tissue disruption, early weight bearing, and improved biomechanical load sharing. Disadvantages of nailing, however, may include limitations in the number of fixation points and planes, inability to stabilize articular fractures, and difficult access to some starting points. Thus, there remains a need for improved plating systems and/or intramedullary systems that provide appropriate stabilization to the bone.

### SUMMARY OF THE INVENTION

To meet this and other needs, and in view of its purposes, the present application provides devices, systems, instruments, and methods for promoting healing and stability for bone fractures. In particular, targeted interlocking plate-nail constructs may be used to treat a wide range of fractures. The plate-nail construct combines the benefits of both locked plating and intramedullary nailing to treat many types of fracture patterns including treating fractures in patients with poor bone quality. The plate-nail construct provides for the load bearing and minimally invasive features of intramedullary nails along with the multiple locking options and rigid fixation of locked plating.

According to one embodiment, a bone stabilization system includes a bone plate, an intramedullary nail, and a plurality of fasteners. The bone plate is configured to be positioned against an exterior surface of a long bone. The bone plate has a plurality of fastener openings defined therethrough. The intramedullary nail is configured to extend through a medullary canal of the long bone. The intramedullary nail defines a plurality of holes therethrough. A first fastener is configured to extend through one of the fastener openings in the bone plate and through one of the holes in the intramedullary nail and into the bone, thereby interlocking the bone plate and the intramedullary nail together. A second fastener is configured to extend through one of the fastener openings in the bone plate and directly into the bone without passing through the intramedullary nail. A third fastener is configured to extend through one of the holes in the intramedullary nail and directly into the bone without passing through the bone plate.

The bone stabilization system may include one or more of the following features. The first fastener may include a locking screw that threads or deforms material in both the bone plate and the intramedullary nail, a locking screw that threads into the bone plate only, a headed or headless locking screw that threads into the nail only, or a non-locking screw that does not lock to the bone plate or the intramedullary nail. The second fastener may include a plate locking screw configured to secure one end of the bone plate into epiphysis of the bone. The bone stabilization system may include a fourth fastener configured to extend through one of the fastener openings in the bone plate, around the intramedullary nail, and directly into bone without passing through the intramedullary nail. The fourth fastener may include a plate locking screw configured to secure the bone plate into diaphysis of the bone. The fourth fastener may be part of a pair of fasteners positioned on opposite sides of the intramedullary nail. The bone stabilization system may include a fifth fastener configured to extend through one of the fastener openings in the bone plate and into the neck and head of the long bone. The first fastener may be part of one set of fasteners configured for interlocking a distal end of the bone plate and the intramedullary nail and another set of fasteners configured for interlocking a proximal end of the bone plate and the intramedullary nail.

According to one embodiment, a targeted plate-nail system includes an intramedullary nail defining a plurality of holes therethrough, a nail insertion handle configured for inserting the intramedullary nail into a medullary canal of a long bone, the nail insertion handle including a handle grip and a coupling portion configured to secure one end of the intramedullary nail, a bone plate having a plurality of fastener openings defined therethrough, a plurality of fasteners configured to extend through the fastener openings in the bone plate, through the holes in the intramedullary nail, or through both, and a nail aiming arm attachable to the nail insertion handle to guide the plurality of fasteners into the bone plate, intramedullary nail, or through both, thereby interlocking the bone plate and the intramedullary nail together.

The targeted plate-nail system may include one or more of the following features. The system may also include a connection shaft attachable to the bone plate. The connection shaft may include one or more datum pins, ball, spheres that enables the orientation of the connection shaft to the bone plate receivable in corresponding datum recesses defined in a top face of the bone plate, and a threaded connection bolt receivable in a threaded opening in the bone plate to thereby secure the connection shaft to the bone plate. The system may also include a drill aiming arm and a drill sleeve attachable to the connection shaft to create an intramedullary nail entry portal. The nail aiming arm may include an arcuate body with opposed end supports for left and right access. Each end support may define one or more targeted hole openings which align with respective fastener openings in the bone plate and holes in the intramedullary nail. The system may also include a proximal aiming arm attachable to the nail aiming arm with one or more connection pins. The proximal aiming arm may define a pocket sized and dimensioned to receive a free end of the end support of the aiming arm, thereby covering the targeted hole openings in the nail aiming arm. The proximal aiming arm may define a long free arm configured to align with the bone plate. The proximal aiming arm may define a plurality of guide hole openings configured to guide the plurality of fasteners into the bone plate only or the bone plate and intramedullary nail.

According to one embodiment, a targeted interlocking plate-nail system includes a bone stabilization implant and a targeting instrument. The bone stabilization implant includes an intramedullary nail configured to extend through a medullary canal of a long bone, a bone plate configured to be positioned against an exterior surface of the long bone, and a plurality of fasteners configured to extend through the bone plate, through the intramedullary nail, or through both the bone plate and the intramedullary nail. The targeting instrument includes a nail insertion handle, a nail aiming arm, and a proximal aiming arm. The nail insertion handle is configured for inserting the intramedullary nail into the bone. The nail aiming arm is configured to guide some of the fasteners into the bone plate and intramedullary nail, thereby interlocking the bone plate and the intramedullary nail together. The proximal aiming arm is configured to guide some of the fasteners through the bone plate and intramedullary nail or into the bone plate only.

The targeted interlocking plate-nail system may include one or more of the following features. The fasteners may include a locking screw that threads or deforms material into both the bone plate and the intramedullary nail, a locking screw that threads into the bone plate only, a headed locking screw that threads into the nail only, or a non-locking screw that does not lock to the bone plate or the intramedullary nail. The stiffness of the bone stabilization implant may be modulated by varying the number, type, and location of fasteners. The bone stabilization implant may be installed using a plate first or nail first approach depending on the fracture type and location.

According to one embodiment, a method of stabilizing a bone fracture includes one or more of the following steps in any suitable order: (1) positioning a bone plate against an exterior surface of a long bone; (2) securing the bone plate to the bone by attaching a first set of fasteners through the bone plate while avoiding an exclusionary zone intended to later receive an intramedullary nail; (3) inserting the intramedullary nail into a medullary canal of the bone with a nail insertion handle due to access provided by the exclusionary zone; (4) attaching a nail aiming arm to the nail insertion handle, the nail insertion handle having an end support with targeted hole openings that align to the bone plate; and (5) inserting a second set of fasteners through the targeted hole openings in the nail aiming arm into bone plate and intramedullary nail, thereby interlocking the bone plate and the intramedullary nail together. The method may further include: (6) attaching a proximal aiming arm to the nail aiming arm with one or more connection pins, the proximal aiming arm defining a plurality of guide hole openings; (7) inserting another set of fasteners through the guide hole openings in the proximal aiming arm into the bone plate and the intramedullary nail, thereby interlocking the bone plate and nail together; (8) inserting another set of fasteners through the guide hole openings in the proximal aiming arm and into the bone plate only such that some of the fasteners positioned through the bone plate only target a neck and head of the long bone and some of the fasteners positioned through the bone plate only target a shaft of the long bone while avoiding the intramedullary nail; and/or (9) inserting a single fastener through one of the targeted hole openings in the nail aiming arm and into the intramedullary nail only. The exclusionary zone may be identified on a surface of the plate, for example, with a rectangle delineating fastener openings through the bone plate nominally pointed toward the intramedullary nail.

According to another embodiment, a method of stabilizing a bone fracture includes one or more of the following steps in any suitable order: (1) positioning a bone plate against an exterior surface of a long bone; (2) securing the bone plate to the bone by attaching a first set of fasteners through the bone plate while avoiding an exclusionary zone intended to later receive an intramedullary nail; (3) inserting the intramedullary nail into a medullary canal of the bone with a nail insertion handle due to access provided by the exclusionary zone; (4) attaching a nail aiming arm to the nail insertion handle, the nail insertion handle having an end support with targeted hole openings that align to the bone plate; (5) inserting a second set of fasteners through the targeted hole openings in the nail aiming arm into bone plate and intramedullary nail, thereby interlocking the bone plate and nail together; (6) attaching a proximal aiming arm to the nail aiming arm, the proximal aiming arm defining a plurality of guide hole openings; (7) inserting a third set of fasteners through the guide hole openings in the proximal aiming arm into the bone plate and the intramedullary nail; (8) inserting a fourth set of fasteners through the guide hole openings in the proximal aiming arm and into the bone plate only; (9) inserting a fifth set of fasteners through the guide hole openings in the proximal aiming arm and into the bone plate only; and (10) inserting a sixth fastener through one of the targeted hole openings in the nail aiming arm and into the intramedullary nail only. The method may further include: (11) after securing the bone plate to the bone, attaching a connection shaft to the bone plate; (12) after attaching the connection shaft to the bone plate, attaching a drill aiming arm and drill sleeve to the connection shaft, wherein the drill sleeve is aligned along a trajectory; (13) after attaching the drill aiming arm and drill sleeve to the connection shaft, drilling an entry portal into the bone by passing a drill bit through the drill sleeve; (14) prior to positioning the bone plate, attaching the connection shaft to the bone plate to install the bone plate; and/or (15) prior to positioning the bone plate, reducing the bone fracture to restore length, alignment, and rotation of the bone. In the case of a femur, the intramedullary nail may be inserted into a distal end of the femur, and the bone plate may sit on a lateral surface of the femur such that a distal end of the bone plate secures the distal end of the femur and a proximal end of the bone plate secures a head of the femur. In the case of a tibia, the intramedullary nail may be inserted into a proximal end of the tibia, and the bone plate may sit on a lateral surface of the tibia such that a proximal end of the bone plate secures the proximal end of the tibia and a distal end of the bone plate secures a shaft of the tibia.

According to yet another embodiment, a method of stabilizing a bone fracture includes one or more of the following steps in any suitable order: (1) obtaining a bone stabilization implant comprising an intramedullary nail, a bone plate, and a plurality of fasteners configured to extend through the bone plate, through the intramedullary nail, or through both the bone plate and the intramedullary nail; and (2) selecting a nail first or plate first workflow depending on fracture type and location, wherein the nail first workflow includes inserting the intramedullary nail into the medullary canal first and subsequently attaching the bone plate to an exterior surface of the bone, and wherein the plate first workflow includes attaching the bone plate to the exterior surface of the bone first and subsequently inserting the intramedullary nail into the medullary canal of the bone. The method may further include: (3) interlocking a distal end of the bone plate and intramedullary nail together with one set of fasteners and interlocking a proximal end of the bone plate and intramedullary nail together with another set of fasteners; and/or (4) adjusting a stiffness of the bone stabilization implant by varying a number, type, or location of fasteners. The plate may identify an exclusionary zone such that when selecting the plate first workflow, fasteners are not entered into the exclusionary zone intended to later receive the intramedullary nail. The plurality of fasteners may include a locking screw that threads into both the bone plate and the intramedullary nail, a locking screw that threads into the bone plate only, a headed locking screw that threads into the nail only, or a non-locking screw that does not lock to the bone plate or the intramedullary nail.

Also provided are kits for the stabilization systems including bone plates of varying sizes and orientations, intramedullary nails of varying sizes and orientations, fasteners including locking fasteners, non-locking, compression fasteners, polyaxial fasteners, fixed angle fasteners, or any other suitable fasteners, drill guides, k-wires, sutures, and other components for installing the same.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 shows a plate-nail construct interlocked with a plurality of screws to increase the construct stiffness according to one embodiment;

FIGS. 2A-2C show cross-sectional and close-up views, respectively, of a locking screw engaged with the intramedullary nail and the plate for maximum construct stiffness according to one embodiment;

FIGS. 3A-3C show cross-sectional and close-up views, respectively, of a locking screw not engaged with the intramedullary nail and engaged with only the plate for increased construct stiffness according to one embodiment;

FIGS. 4A-4C show cross-sectional and close-up views, respectively, of a headed locking screw engaged with the intramedullary nail and compressed with the plate for reduced construct stiffness according to one embodiment;

FIGS. 5A-5C show cross-sectional and close-up views, respectively, of a non-locking screw not engaged with the intramedullary nail and compressed with the plate for minimum construct stiffness according to one embodiment;

FIGS. 6A-6C show a plate first workflow of inserting a plate and using a connection shaft to help insert the plate against the bone according to one embodiment;

FIGS. 7A-7E show inserting locking screws through the plate and into bone while avoiding the exclusionary zone where the intramedullary nail is intended to be placed later according to one embodiment;

FIGS. 8A-8C show attachment of a connection shaft with a threaded connection bolt to the plate via datum alignment features according to one embodiment;

FIGS. 9A-9B show attachment of a drill sleeve to the connection shaft and alignment of the drill sleeve to the intramedullary nail start point according to one embodiment;

FIG. 10 shows a drill positionable through the entry drill sleeve according to one embodiment;

FIGS. 11A-11B show insertion of the intramedullary nail into the bone with a nail insertion handle according to one embodiment;

FIG. 12 shows attachment of a nail aiming arm to the nail insertion handle according to one embodiment;

FIGS. 13A-13B show insertion of three locking screws to interconnect the plate and the intramedullary nail through the nail aiming arm according to one embodiment;

FIG. 14 shows attachment of a proximal aiming arm to the nail aiming arm, which is secured by two connection pins according to one embodiment;

FIGS. 15A-15B show insertion of two proximal locking screws targeted through the proximal aiming arm to interconnect the plate and the nail according to one embodiment;

FIGS. 16A-16B show insertion of a pair of prophylactic screws inserted through the aiming arm and through the plate and into the femoral neck and head of the bone according to one embodiment;

FIGS. 17A-17C show locking screws inserted through the aiming arm, into the plate, and around the nail according to one embodiment;

FIGS. 18A-18B show the final plate-nail construct for stabilizing a femur bone according to one embodiment with the instrumentation removed;

FIGS. 19A-19B show the final plate-nail construct according to one embodiment with the bone omitted for clarity;

FIGS. 20A-20B show a nail first workflow according to one embodiment including inserting the intramedullary nail through a standard entry portal after reducing the fracture;

FIG. 21 shows inserting a medial oblique locking screw through the nail aiming arm according to one embodiment;

FIG. 22 shows removing the nail aiming arm and inserting a plate adjacent to the bone with a connection shaft according to one embodiment;

FIG. 23 shows removing the connection shaft and assembling the nail aiming arm to the insertion handle according to one embodiment;

FIG. 24 shows inserting three interlocking screw through the plate and nail using the nail aiming arm according to one embodiment;

FIGS. 25A-25B show removing the nail aiming arm and adding distal plate locking screws to the construct according to one embodiment;

FIG. 26 shows attaching a proximal aiming arm and adding additional locking screws to control construct stiffness according to one embodiment;

FIGS. 27A-27B show a proximal tibia plate-nail construct according to one embodiment;

FIGS. 28A-28B show the proximal tibia plate-nail construct of FIGS. 27A-27B with the bone not shown for clarity;

FIGS. 29A-29B show a proximal femur plate-nail construct according to one embodiment; and

FIGS. 30A-30B show the proximal femur plate-nail construct of FIGS. 29A-29B with the bone omitted for clarity.

### DETAILED DESCRIPTION OF THE INVENTION

Embodiments of the disclosure are generally directed to devices, systems, instrument, and methods for promoting healing and stability for bone fractures. An interlocked targeted plate-nail construct combines the benefits of locked plating and intramedullary nailing when treating a wide range of fracture types and fracture patterns. The plate-nail construct may be especially effective at treating fractures in patients with poor bone quality, such as osteoporotic bone. The plate-nail construct provides the load bearing and minimally invasive features of intramedullary nails with the multiple locking options and rigid fixation of locked plating. The plate-nail construct may be interlocked (screws pass through both plate and nail) or independent (plate screws do not interface with nail and/or nail screws do not interface with the plate). In both cases, the surgeon has the option to modulate the stiffness of the construct by adding or reducing the number of screws, using locking or non-locking screws, increasing or decreasing the distance between the locking screws, and/or choosing between interlocking or independent implants. The numerous options for controlling construct stiffness gives the plate-nail construct an advantage in treating a wide range of fracture types in healthy, young bone, as well as in osteoporotic bone. The ability to increase stiffness and load sharing with a plate-nail construct may be especially beneficial in patients with highly comminuted fractures requiring numerous points of fixation and in patients with poor bone quality that require unique locking options that allow the surgeon to utilize screws in locations where bone quality is optimum.

In cases of intra-articular fractures, a bone plate may be used to create a very rigid construct at the articular block to promote primary healing with absolute stability. An intramedullary nail in the bone canal provides the biomechanical advantage of being closer to the mechanical axis than a plate that is secured to the outside of the bone. The further the implant is from the mechanical axis, the greater the bending moment applied to the implant. The surgeon may add an intramedullary nail to the fractured bone to allow the patient to bear weight earlier and maintain mobility and independence during recovery. Multiple interlocking options between the plate and nail allow the surgeon to create implant constructs that are unique to the patient's needs. The construct may be modulated from absolute stiffness and rigidity for primary healing to dynamized motion to promote callus formation and secondary bone healing.

The plate-nail construct may be applied by the surgeon using a plate first or nail first approach depending on the fracture type and location. The plate-nail construct allows the freedom to apply the implants in the order that treats the fracture most effectively by utilizing the unique instrumentation in this system. The intramedullary nail and plate implants may be interconnected using one or more insertion instruments. The insertion instruments may be used alone to insert an individual implant or may be used together to interconnect the nail and plate. The joint used to interconnect the two implants may also align the implants so that the appropriate locking holes are aligned and may be interlocked if desired. Locking and/or non-locking screws may be used to interlock the nail and plate construct to further modulate construct stiffness. The interlocked targeted plate-nail constructs may be useful on many anatomic regions of the body including long bones, such as the tibia, femur, and humerus.

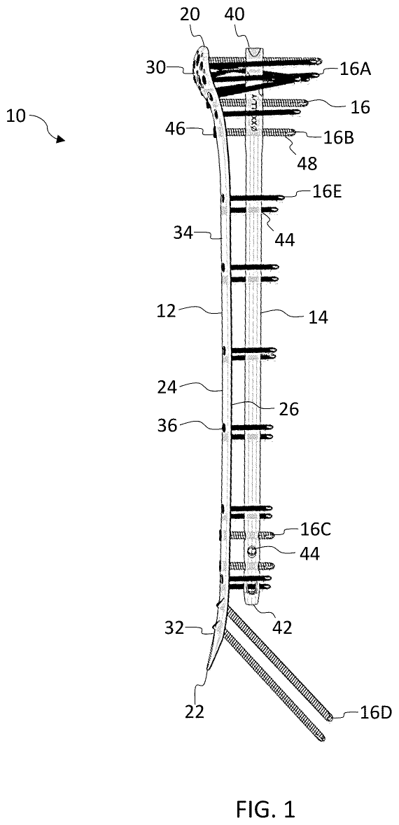

Referring now to FIG. 1, an implant stabilization system or plate-nail construct 10 is shown according to one embodiment. The plate-nail construct 10 is comprised of a bone plate 12 configured to be positioned against an outside face of a long bone and an intramedullary nail 14 configured to be positioned through the medullary canal of the bone 2. A plurality of bone fasteners 16 are configured to pass through the plate 12 and into the bone 2, through the plate 12 and intramedullary nail 14 to interlock them, and/or through the intramedullary nail 14 and into the bone. When one or more fasteners 16 are interconnected between the plate 12 and nail 14, the interlocking configuration increases the stiffness and rigidity of the overall construct 10. The construct 10 includes multiple interlocking options between the plate 12 and nail 14, which allow for customized implant constructs that are unique to the patient's needs.

FIG. 1 depicts one embodiment of a femur bone plate 12 configured to sit on a lateral surface of a femur bone 2. The femur 2 is a long bone in the leg or thigh that runs from the hip to the knee. The femur 2 includes the epiphyses (e.g., the wider or rounded end portions), the diaphysis (e.g., midsection or shaft), and the metaphysis (e.g., where the bone flares between the diaphysis and the epiphysis) of the long bone. The proximal epiphysis includes the neck and head of the femur, which participate in formation of the hip joint, and the distal epiphysis includes the medial and lateral condyles, which participate in formation of the knee joint. The bone fasteners 16 may be configured to secure the length of the femur bone 2 including a distal end or lower end of the femur, a proximal end or head portion of the femur 2, and points in between. The bone plate 12 spans the bone fracture(s) to hold the bone fragments together, allowing the bone 2 to heal in the correct alignment. Although construct 10 is generally described with reference to stabilizing the distal femur, it will be appreciated that the stabilization systems described herein may be used or adapted to be used for the fixation of other areas or other long bones as well, such as a distal tibia, a proximal tibia, a proximal humerus, a distal humerus, a clavicle, a fibula, an ulna, a radius, bones of the foot, bones of the hand, or other suitable bone or bones. The bone plates 12 may come in various shapes, sizes, and designs tailored for specific bones and types of fractures.

In the case of distal femur bone plate 12, the plate 12 includes a flat thin implant that extends from a first end or distal end 20 configured to be positioned proximate to a distal portion of the femur (e.g., adjacent the lateral epicondyle) to a second end or proximal end 22 configured to be positioned toward a proximal portion of the femur (e.g., opposite the head of the femur). The plate 12 includes a top surface 24 and an opposite, bottom surface 26 configured to contact adjacent bone 2. The top and bottom surfaces 24, 26 are connected by opposite side surfaces extending from the first to second ends 20, 22 of the plate 12. The bone plate 12 may be curved, contoured, straight, or flat. The bottom surface 26 of the plate 12 may include anatomic contouring configured to follow the best approximation of average long bone anatomy.

The bone plate 12 may include a distal portion 30, a proximal portion 32, and an elongated central portion or shaft portion 34 between the distal and proximal portions 30, 32 extending along a longitudinal axis. The distal portion 30 of plate 12 may have a head portion that is contoured to match a particular bone surface, such as the epiphysis or metaphysis of the bone 2. In some cases, the plate 12 may form an L-shape, T-shape, Y-shape, etc., with the shaft portion 34 or any other appropriate shape to fit the anatomy of the bone to be treated. In one embodiment, the distal portion 30 may include an enlarged rounded head extending from the shaft portion 34. The bottom surface 26 of the enlarged head portion 30 may be configured to contact the lateral epicondyle of the femur 2. The shaft portion 34 of the plate 12 is configured to contact the diaphysis, shaft, or central part of the femur bone 2. The elongated portion 34 may terminate at the proximal end 22 with a taper such that it has a width and/or thickness less than the remainder of the elongated shaft 34. The tapered end 22 may facilitate insertion of the plate 12, for example, through a percutaneous incision.

The bone plate 12 includes one or more through openings 36 each configured to receive a bone fastener 16 therethrough. The fastener openings 36 extend through the body of the plate 12 from the top surface 24 to the bottom surface 26. The fastener openings 36 may include cylindrical openings, conical openings, elongated openings, threaded openings, textured openings, non-threaded and/or non-textured openings, and the like. The openings 36 may allow for locking of the fastener 16 to the plate 12 or may allow for movement and dynamic compression of the bone. The plate 12 may comprise any suitable number of fastener openings 36 in any suitable configuration. These openings 36 allow surgeons flexibility for fastener placement, based on preference, anatomy, and fracture location. Surgeons may have differing opinions as to the number, location, and types of fasteners 16. Further, complexity of fracture location and shape makes having as many locations for fasteners 16 as possible necessary to treat the fracture(s). The design of plate 12 offers surgeons a versatile method to achieve higher accuracy in placement of the fasteners 16.

The intramedullary nail 14 is a long rod or stem that extends from a first end or distal end 40 to a second end or proximal end 42. The nail 14 is configured to extend longitudinally within the intramedullary canal of the femur bone 2. In the case of insertion through the distal femur, the proximal end 42 of the nail 14 extends proximally through the medullary canal and the distal end 40 of the nail 14 is located in the distal end of the femur. The nail 14 may be configured as a cylindrical shaft or tubular rod, however, the shaft may be configured with any geometrical cross-dimensional shape (e.g., rectangular, oval, elliptical, oblong, polygonal, or the like) that suits the intramedullary canal. The nail 14 may be substantially straight or bent to mimic the natural anatomical curvature of the long bone. The nail 14 defines a plurality of through openings or holes 44 configured for receiving the fasteners 16 described herein. Each of the plurality of fastener holes 44 may have an entry point and an exit point thought the nail 14. The fastener holes 44 may be threaded or textured (e.g., to receive locking fasteners 16) or non-threaded/non-textured (e.g., to receive compression fasteners 16). The nail 14 may come in various shapes, sizes, and designs tailored for specific bones and types of fractures.

The plate 12 and/or nail 14 may be configured to receive bone fasteners 16. The fasteners 16 may include locking fasteners, non-locking fasteners, or any other fasteners known in the art. The fasteners 16 may comprise bone screws or the like. The fasteners 16 may be cannulated such that they may be guided into place over guide wires. The fasteners 16 may also include other fasteners or anchors configured to be secured or engaged with bone, such as nails, spikes, staples, pegs, barbs, hooks, or the like. In some embodiments, the fasteners 16 may include fixed and/or variable angle bone screws. The fastener 16 may include a head portion 46 and a threaded shaft portion 48 configured to engage bone. In the case of a locking fastener 16, the head portion 46 may include a textured area, such as threads, around its outer surface sized and configured to engage with the fastener opening 36, for example, with corresponding threads in the opening 36 in order to lock the fastener 16 to the plate 12. In the alternative, for a non-locking fastener 16, the head portion 46 may be substantially smooth to allow for dynamic compression of the bone 2. Similarly, the fasteners 16 may have a threaded shaft portion 48 configured to engage corresponding threads in the nail 14 to lock the fastener 16 to the nail 14. Alternatively, the shaft portion 48 may be received through opening 44 in nail 14 while not engaging directly with threads in opening 44, thereby not directly locking the shaft 48 of fastener 16 to the nail 14.

The bone plates 12 and intramedullary nails 14 may be available in a variety of lengths, widths, and styles based on the anatomy of the patient. The plates 12 and nails 14 may be configured in both left and right designs, in a mirrored configuration, in order to address the anatomy of both the left and right sides of the patient. The systems may be adapted to secure small or large bone fragments, single or multiple bone fragments, or otherwise secure one or more fractures. In particular, the systems may include a series of trauma plates 12, intramedullary nails 14, and screws 16 designed for the fixation of fractures and fragments in diaphyseal, metaphyscal, and epiphyseal bone. Different systems may be used to treat various types and locations of fractures.

The bone plate 12 and/or intramedullary nail 14 may be comprised of titanium, stainless steel, cobalt chrome, carbon composite, plastic or polymer—such as polyetheretherketone (PEEK), polyethylene, ultra high molecular weight polyethylene (UHMWPE), resorbable polylactic acid (PLA), polyglycolic acid (PGA), combinations or alloys of such materials or any other appropriate material that has sufficient strength to be secured to and hold bone, while also having sufficient biocompatibility to be implanted into a body. Similarly, the fasteners 16 may be comprised of titanium, cobalt chrome, cobalt-chrome-molybdenum, stainless steel, tungsten carbide, combinations or alloys of such materials or other appropriate biocompatible materials. Although the above list of materials includes many typical materials out of which bone plates, intramedullary nails, and bone fasteners are made, it should be understood that the bone plates, intramedullary nails, and fasteners comprised of any appropriate material are contemplated.

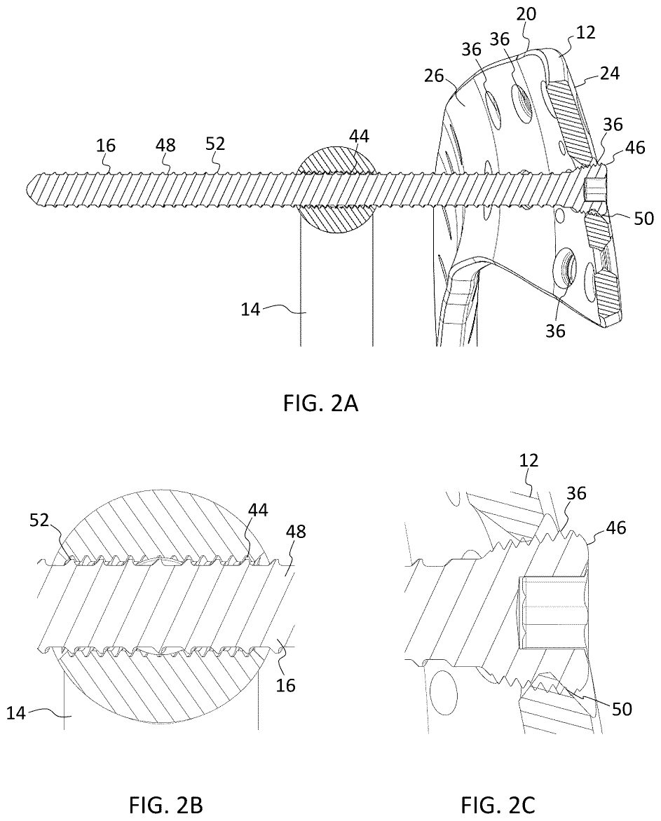

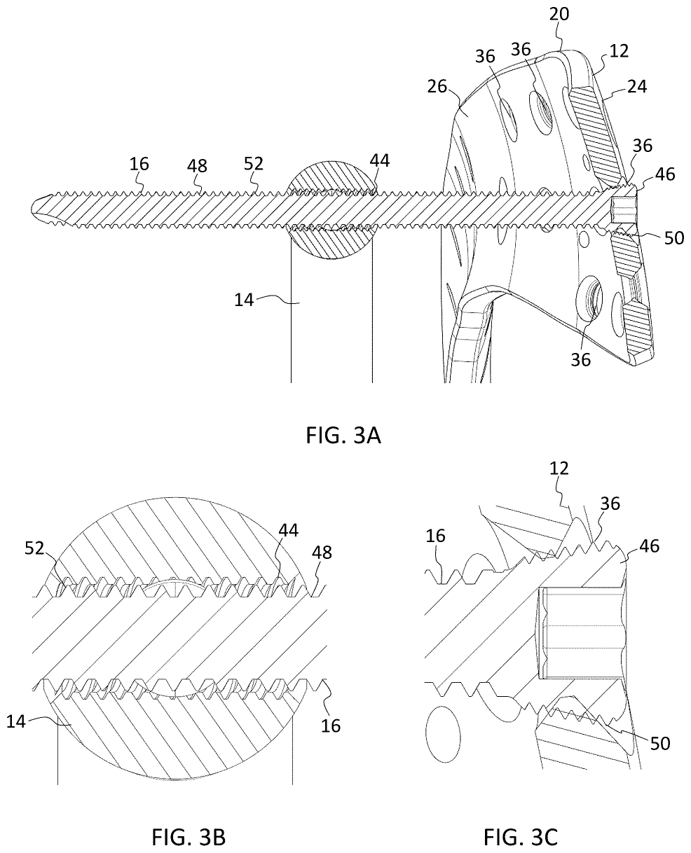

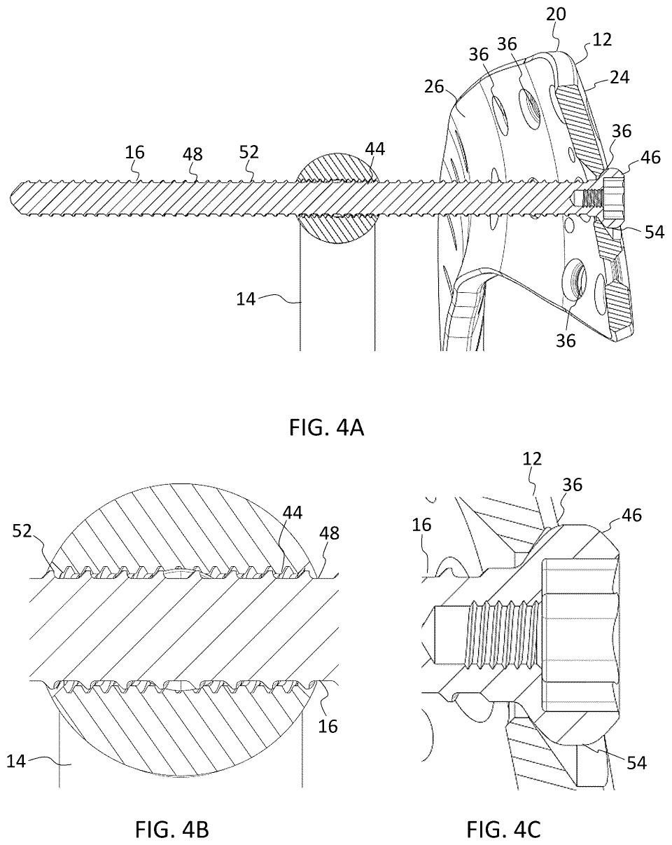

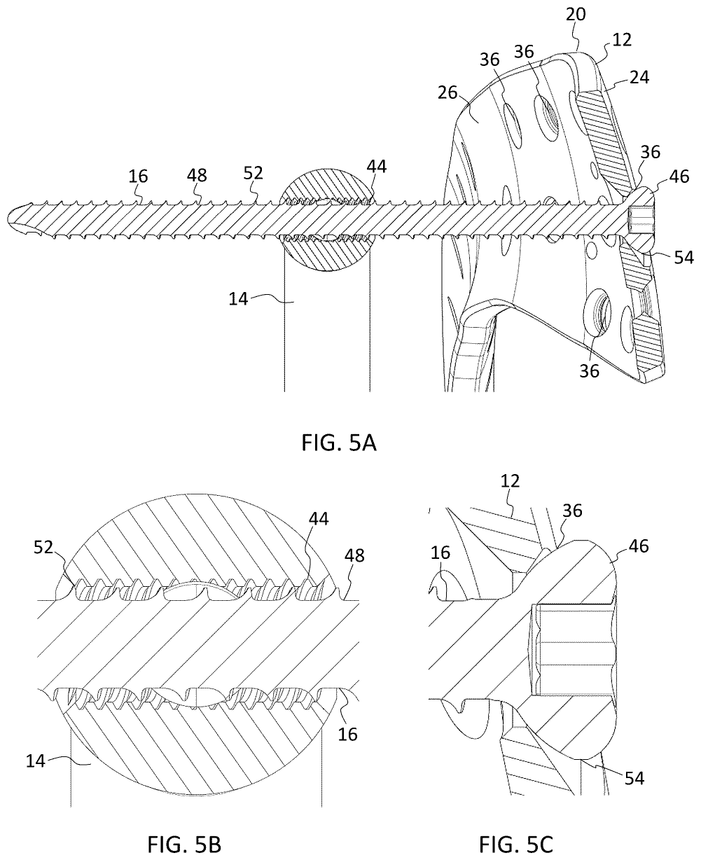

Turning now to FIGS. 2A-5C, examples of connections between the plate 12, intramedullary nail 14, and fasteners 16 are described in further detail. The types of connections between the plate 12, nail 14, and fasteners 16 allow the surgeon additional control over construct stiffness. FIGS. 2A-2C show one example of a locking screw 16 threaded or deforms material into both the plate 12 and intramedullary nail 14 to provide a maximum amount of construct stiffness. FIGS. 5A-5C show one example of a non-locking screw through the plate 12 and intramedullary nail 14 to provide a minimum amount of construct stiffness. FIGS. 3A-3C show one example of a locking screw that threads or deforms material into the plate 12 only and FIGS. 4A-4C show one example of a headed locking screw that threads into the nail 14 only, thereby having construct stiffnesses between the extremes. Thus, the construct stiffness ratings ordered from highest to lowest are: (1) locking screw threads or deforms material into both plate 12 and nail 14 (FIGS. 2A-2C); (2) locking screw threads or deforms material into plate 12 only (FIGS. 3A-3C); (3) headed locking screw threads into nail 14 only (4A-4C); and (4) non-locking screw positioned through plate 12 and nail 14 (FIGS. 5A-5C).

FIGS. 2A-2C depict an example of locking screw 16 with a conical head 46 having a partially or fully threaded portion 50 configured to lock the screw 16 to the plate 12. As best seen in FIG. 2C, the locking screw head 46 deforms threads into the polyaxial hole 36 of the plate 12, thereby securing the screw 16 to the plate 12. As best seen in FIG. 2B, the shaft 48 of the screw 16 has exterior threads 52 configured to lock the screw 16 to the intramedullary nail 14. The locking screw threads 52 of shaft 48 engage with the corresponding threads of threaded hole 44 through intramedullary nail 14, thereby securing the screw 16 to the intramedullary nail 14. This interlocked configuration, locking screw threads into both the plate 12 and the intramedullary nail 14, provides the highest or maximum amount of construct stiffness for the plate 12 and intramedullary nail 14.

FIGS. 3A-3C depict an example of locking screw 16 that threads or deforms material into the plate 12 only. Similar to FIG. 2C, the locking screw 16 has a conical head 46 with a threaded exterior 50 configured to lock the screw 16 to the opening 36 through plate 12. As best seen in FIG. 3C, the threaded locking screw head 46 deforms threads into the polyaxial hole 36 of the plate 12, thereby securing the screw 16 to the plate 12. FIG. 3B shows the shaft 48 of the screw 16 has exterior threads 52 that do not engage with the intramedullary nail 14. This configuration may occur, for example, when the outer diameter of the fastener 16 is smaller than the inner diameter of the opening 44 through nail 14. In other words, when the fastener 16 is positioned through opening 44 in nail 14, the locking screw threads 52 on the fastener 16 do not interface with the threaded hole 44 in nail 14. This interlocked configuration, where the locking screw 16 threads into plate 12 only, provides an increased construct stiffness for the plate 12 and nail 14 assembly, which providing some dynamic movement with the nail 14.

FIGS. 4A-4C depict an example of a headed locking screw 16 that threads into the nail 14 only. In this case, the headed locking screw 16 has a compression head 46 with a smooth conical, tapered, or rounded exterior surface 54 configured to compress the screw 16 to the plate 12. As best seen in FIG. 4C, as the screw 16 is threaded into bone, the compression screw head 46 compresses against the plate 12, thereby resulting in compression of the bone fragments beneath the plate 12 ensuring tight apposition and promoting bone healing. FIG. 4B shows the shaft 48 of the screw 16 has exterior threads 52 that engage with the threaded opening 44 through the intramedullary nail 14, thereby locking the screw 16 to the intramedullary nail 14. The locking screw threads 52 engage with the corresponding threads of threaded hole 44 through intramedullary nail 14, thereby securing the screw 16 to the nail 14. This interlocked configuration with a headed locking screw 16 that threads into the nail 14 only provides a lesser construct stiffness for the plate 12 and nail 14 assembly.

FIGS. 5A-5C depict an example of a non-locking screw 16 positioned through the plate 12 and intramedullary nail 14. Similar to FIG. 4C, the headed locking screw 16 shown in FIG. 5C is a compression head 46 with a smooth conical, tapered, or rounded exterior surface 54. When tightened, the compression screw head 46 compresses against the plate 12, thereby resulting in compression of the bone fragments beneath the plate 12. Similar to FIG. 3B, the shaft 48 of the screw 16 has exterior threads 52 that do not engage with the intramedullary nail 14. As shown in FIG. 5B, this configuration may occur when the outer diameter of the fastener 16 is reduced relative to the inner diameter of the opening 44 through nail 14. In other words, when the fastener 16 is positioned through opening 44 in nail 14, the screw threads 52 on the fastener 16 do not interface with the threaded hole 44 in nail 14. This non-locked configuration provides a minimum construct stiffness for the plate 12 and nail 14 assembly, with less stiffness than any other configuration. This non-locking configuration also provides the most amount of dynamic movement between the plate 12 and nail 14.

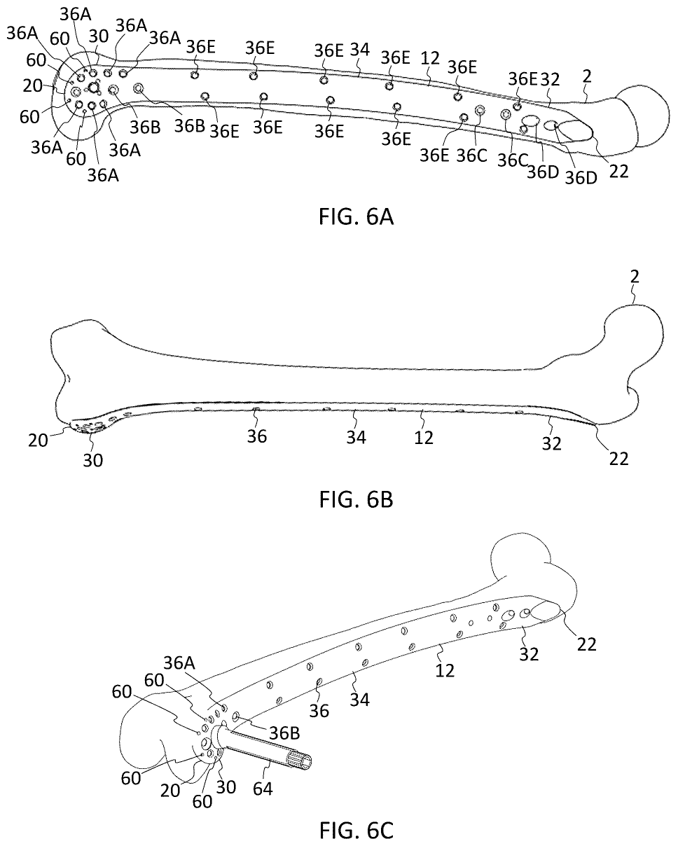

Turning now to FIGS. 6A-17C, a plate first workflow for installing an interlocked targeted nail-plate construct 10 is shown according to one embodiment. Depending on the fracture type and location, a surgeon may select a plate first approach whereby the plate 12 is installed first, some fasteners 16 are secured to the plate 12 and into the bone while maintaining an exclusionary window or keep out zone 62 for the intramedullary nail 14, the intramedullary nail 14 is subsequently installed, and then additional fasteners 16 are added through the plate 12 and nail 14, through the plate 12 only, and/or through the nail 14 only.

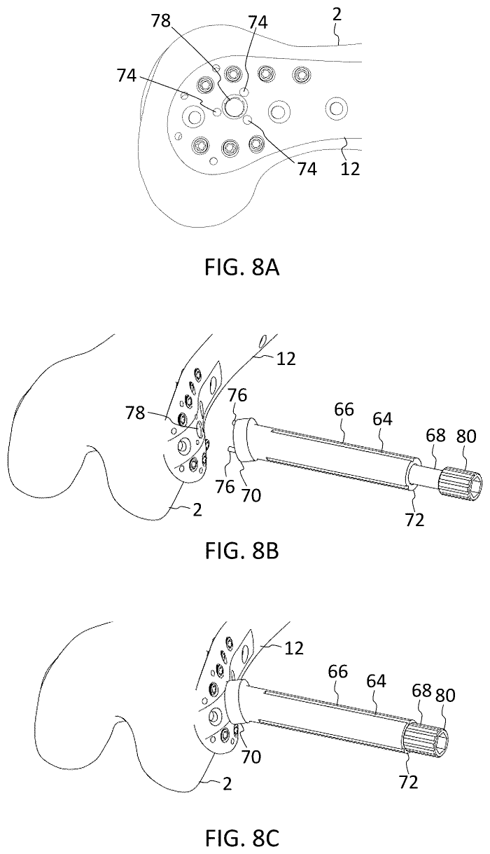

With further emphasis on FIGS. 6A-6C, a bone plate 12 may be placed adjacent to an outer surface of the bone 2. Before the plate 12 is positioned, the fracture may be reduced under fluoroscopy or through an open incision. The bone fragments may be positioned to restore length, alignment, and rotation. In this embodiment, the plate 12 is a femur plate, which may be positioned against a lateral aspect of the femur bone 2. The femur plate 12 may be inserted through a small distal incision. The plate 12 may be installed with a connection shaft 64, which is described in more detail with respect to FIG. 8B. When the plate 12 is positioned against the femur bone, the distal end 20 of the plate 12 may be placed against the lateral epicondyle of the femur and the proximal end 22 of the plate 12 may be placed toward the headed end of the femur. Although a specific type of plate and placement is shown in this embodiment, it will be appreciated that the type of plate and placement may be selected based on the surgeon's preference to treat the fracture type and location of a specific patient.

The plate 12 may be provisionally held in position using k-wires (not shown). The plate 12 may include a plurality of openings 60 configured to receive one or more k-wires. The k-wire holes 60 may comprise small diameter holes (e.g., having a diameter significantly smaller than the fastener openings 36). The k-wire holes 60 may be located in the distal portion 30 of the plate 12, for example, around the perimeter of the enlarged rounded head, or at other suitable locations to allow preliminary placement of the plate 12 against the bone 2 and/or to aid in reduction of the fracture.

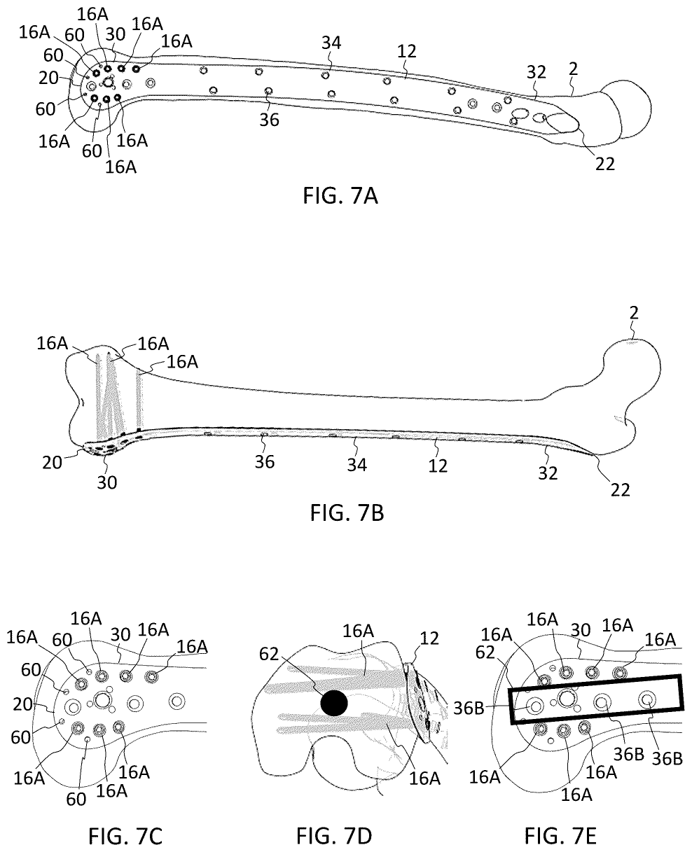

With further emphasis on FIGS. 7A-7E, a plurality of bone fasteners 16 may be inserted through the plate 12 and into the bone based on the fracture pattern and location. In this embodiment, the bone fasteners 16 includes a first set of plate locking screws 16A positioned through fastener openings 36B in the distal portion 30 of the femur plate 12. The distal plate locking screws 16A may have a threaded head 46 (as shown in FIG. 2C) to lock screw 16A to the plate 12. The distal plate locking screws 16A may be nominally aimed medially and/or toward the condyles or in any suitable orientation determined by the surgeon.

If intended to be used with intramedullary nail 14, the distal plate locking screws 16A are preferably inserted while respecting and avoiding an exclusionary zone or nail keep out zone 62. As best seen in FIGS. 7D and 7E, the exclusionary zone or keep out zone 62 may include fastener openings 36B for a second set of plate and nail locking screws 16B. The keep out zone 62 may be visualized as a black circle (axial view in FIG. 7D) for the future placement of the intramedullary nail 14. The keep out zone 62 may be identified on the surface of the plate 12 with, for example, a rectangle (lateral view in FIG. 7E) delineating openings 36B for interlocking plate 12 and nail 14 at a later time. Alternatively, the keep out zone 62 may be marked on the plate 12 with indicators, cutouts, radiopaque markers, or the like. In this manner, the distal locking screws 16A are inserted carefully around the nail keep out zone 62 such that the intramedullary nail 14 may be later introduced into that area without intercepting plate locking screws 16A.

After the plate 12 is secured to the bone 2 with plate locking screws 16A, the bone 2 is prepared for intramedullary nailing. With further emphasis on FIGS. 8A-8C. a connection shaft assembly 64 is attachable to the plate 12. The connection shaft assembly 64 may include a connection shaft 66 with a threaded connection bolt 68 extending therethrough. The connection shaft 66 includes a hollow tube with a distal end 70 configured to attach to the plate 12 and a proximal end 72 configured to receive the connection bolt 68. The plate 12 may define one or more datum alignment features, such as recesses or openings 74 configured to receive corresponding datum pins 76 or spheres or other alignment features extending from the distal end 70 of the connection shaft 66. In one embodiment, three datum openings 74 may be equidistantly spaced about a central threaded opening 78, which is configured to receive one end of the threaded connection bolt 68. After the datum pins 76 are aligned with the respective openings 74, the connection bolt 68 may be positioned through connection shaft 66 and threaded into bolt opening 78, thereby affixing and orienting the connection shaft 66 to the plate 12. The connection bolt 68 may have a proximal tool engagement end 80 configured to interface with a handle or tool to facilitate rotation of the connection bolt 68. After the threaded connection bolt 68 threads into bolt opening 78, the connection shaft assembly 64 is temporarily rigidly affixed to the plate 12.

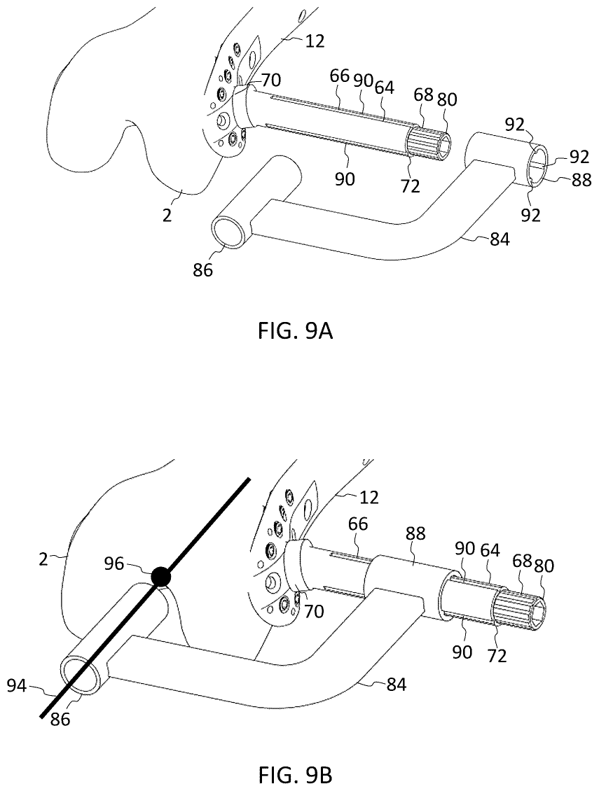

The connection shaft 66 may be used to control the orientation of the drill aiming arm 84 and drill sleeve 86 in relation to the plate 12. With further emphasis on FIGS. 9A-9B, the intramedullary nail entry drill sleeve 86 is attachable to the connection shaft 66 to create an intramedullary nail entry portal. One end of the drill aiming arm 84 includes an attachment sleeve 88 configured to slide over the connection shaft 66. The attachment sleeve 88 includes a tubular body having a through opening configured to accept the connection shaft 66. The connection shaft 66 may define one or more alignment grooves 90 configured to mate with corresponding protruding tabs 92 within the through opening of sleeve 88 through a sliding interface. For example, three mating grooves 90 and tabs 92 may be arranged equidistantly about the shaft 66. It will be appreciated that any suitable number, type, and configuration of interface may be used to connect the aiming arm 84 to the connection shaft 66. The aiming arm 84 may be bent such that the connection between the attachment sleeve 88 and connection shaft 66 create an entry portal trajectory relative to plate position. The drill sleeve 86 may be positioned at the opposite end of the drill aiming arm 84. The drill sleeve 86 may have a tubular body with a through opening configured to receive a drill therethrough. As best seen in FIG. 9B, once assembled, the drill sleeve 86 is aligned along a trajectory 94 for an optimal intramedullary nail start point 96 into the distal end of the femur 2.

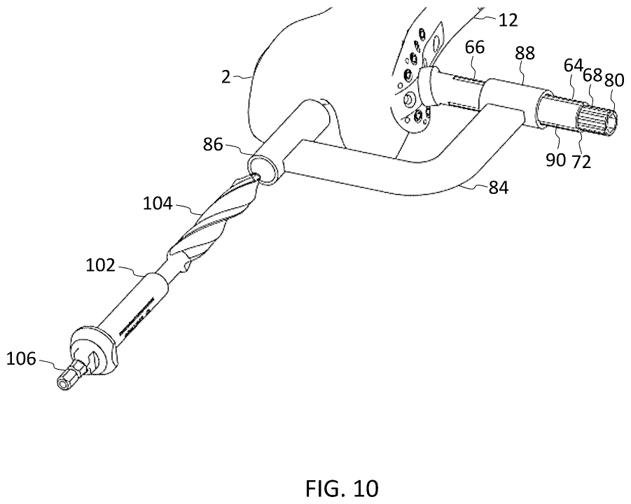

With further emphasis on FIG. 10, an entry portal for the intramedullary nail 14 may be created with an entry drill 102. The entry drill 102 may include a drill bit 104 and an attachment interface 106. The drill bit 104 may have a body with a spiral flute configured to remove bone chips during the drilling process. The attachment interface 106 may be configured to securely couple the drill 102 to a suitable handle or handpiece (e.g., powered) for rotating the drill bit 104 and controlling drilling into the bone 2. The drill bit 104 may be positioned through the entry drill sleeve 86 and into the start point 96 to create a canal of the desired depth and diameter for the intramedullary nail 14. The canal may be further reamed to achieve the appropriate size for accepting the intramedullary nail 14.

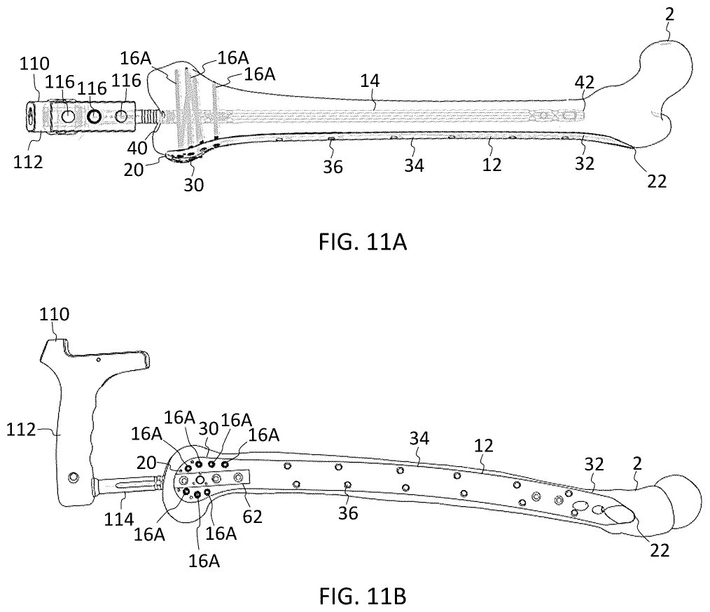

Turning now to FIGS. 11A-11B, the intramedullary nail 14 is inserted through the entry portal and into the medullary canal of the femur bone 2. The intramedullary nail 14 may be inserted with an intramedullary nail insertion handle 110. The insertion handle 110 may include a handle grip 112 and a coupling portion 114 configured to be secured to the intramedullary nail 14. The coupling portion 114 may include a tip with a threaded connector configured to attach to the distal end 40 of the intramedullary nail 14. It will be appreciated that any suitable coupling mechanisms may be employed. The handle grip 112 may include a flat upper body with one or more bores 116 defined therein for aligning and/or securing a nail aiming arm 120 to the insertion handle 110. The bores 116 may include threaded and/or non-threaded bores. The non-threaded bores 116 may be configured to receive connection posts of the aiming arm 120 for aligning the nail aiming arm 120. The threaded bore 116 may be configured to receive a thumb screw 122 of the aiming arm 120 for temporarily attaching the nail aiming arm 120 to the insertion handle 110.

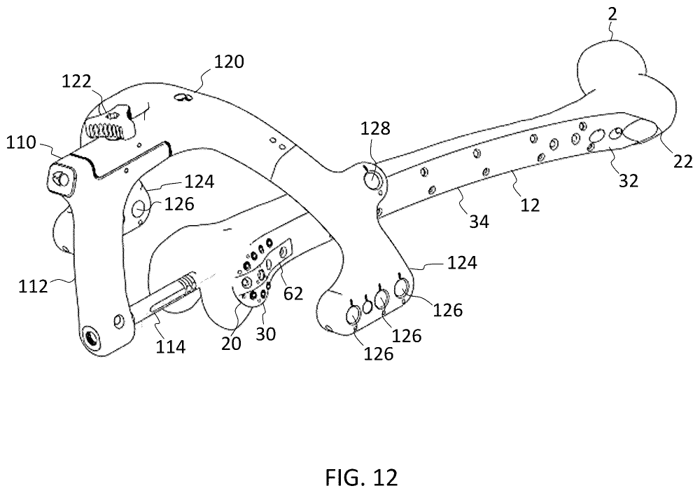

As best seen in FIG. 12, the nail aiming arm 120 is attachable to the nail insertion handle 110 to guide additional bone fasteners 16 into the plate 12 and intramedullary nail 14. The nail aiming arm 120 may attach to the top of the handle grip 112 of the insertion handle 110 with thumb screw 122, for example, which threadedly engages threaded bore 116. The nail aiming arm 120 may be used to install distal locking screws 16B into both the plate 12 and intramedullary nail 14. The nail aiming arm 120 sets the trajectory of the locking screws 16B to interface with the distal openings 36B through the plate 12 and distal openings 44 through the nail 14. The nail aiming arm 120 may include a t-shaped arcuate body which extends between opposed end supports 124. The opposed end supports 124 may extend for left and right access, in a mirrored configuration, in order to address the anatomy of both the left and right sides of the patient. Each end support 124 defines one or more targeted hole openings 126, which align the respective fastener openings 36B, 44 in the plate 12 and nail 14. The targeted hole openings 126 may be aligned in series, for example, along the same plane, to access consecutive openings 36B, 44 in the plate 12 and the nail 14, respectively. The end support 124 may further define a separate targeted opening 128 protruding proximally on each mirrored end support 124. The respective targeted openings 128 may provide for medial oblique or lateral access to the distal portion of the femur and intramedullary nail 14. The guide openings 126, 128 may be configured to support respective guide sheaths (not shown) used to protect the soft tissue during the drilling process.

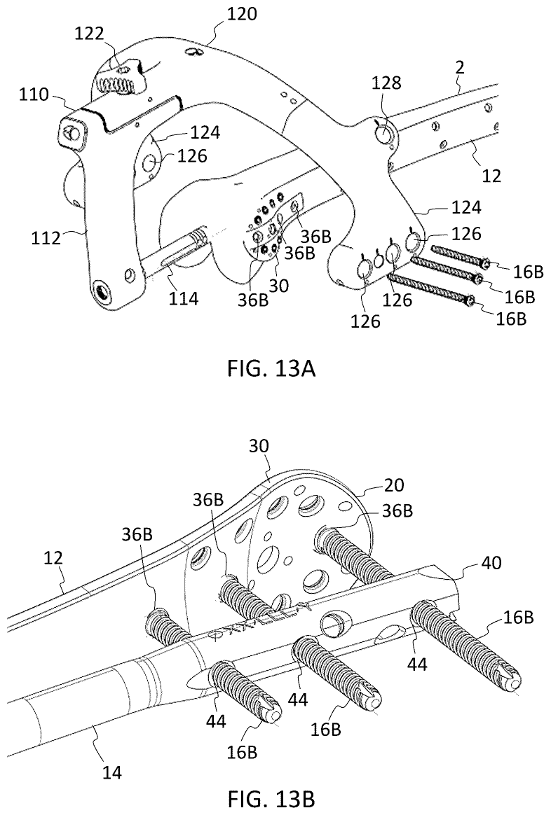

With further emphasis on FIGS. 13A-13B, the free end of each end support 124 of the aiming arm 120 defines three guide hole openings 126. In this manner, up to three interlocking screws 16B may be inserted through both the distal portion 30 of plate 12 and into the distal end 40 of nail 14, thereby interlocking the distal side of the plate 12 and nail 14 together. Due to prior observance of the exclusionary zone or keep out zone 62, the plate and nail locking screws 16B may now be introduced into this area and the screws 16B are easily inserted into the plate 12 and nail 14 without intercepting the first set of plate screws 16A. As shown in FIG. 13A, the distal interlocking screws 16B may be aligned with the guide hole openings 126 in the aiming arm 120 to enter the respective openings 36B through the distal portion 30 of the plate 12. The interlocking screws 16B may include screws of varying lengths or types, for example, as described in FIGS. 2A-5C. FIG. 13B shows the interlocking screws 16B through the plate 12 and intramedullary nail 14 with the plate locking screws 16A and bone omitted for clarity. The plate and nail interlocking screws 16B may thread into both the plate 12 and intramedullary nail 14, may thread only into the plate 12 and pass through the nail holes 44 with clearance, or may thread into the nail 14 and lag the plate 12 to the bone 2. The quantity and type of interlocking screws 16B determine the final construct stiffness.

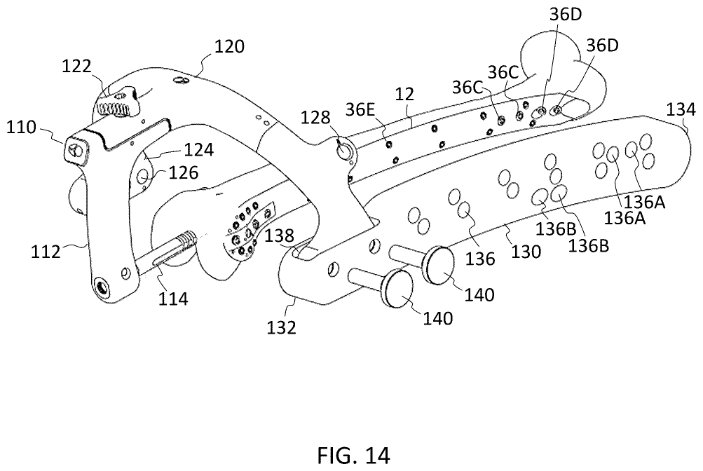

Turning now to FIG. 14, a proximal aiming arm 130 is attached to the nail aiming arm 120. The proximal aiming arm 130 is a long free arm extending from a distal end 132 to a proximal end 134. In this case, the proximal aiming arm 130 may extend proximally to generally run parallel to the plate 12. The proximal aiming arm 130 defines a plurality of guide hole openings 136 configured to install additional screws 16 into the plate 12 and/or nail 14. The distal end 132 of the proximal aiming arm 130 may be attached to the end support 124 of the aiming arm 120. For example, the proximal aiming arm 130 may define a pocket 138 sized and dimensioned to receive the free end of one end support 124 of the aiming arm 120. When the end support 124 is recessed into the pocket 138, the body of proximal aiming arm 130 covers the targeted hole openings 126 in the nail aiming arm 120. One or more connection pins 140 may be used to secure the proximal aiming arm 130 to the nail aiming arm 120. For example, a pair of connection pins 140 having enlarged heads may be positioned through connection holes in the face of the proximal aiming arm 130 and into contact with the nail aiming arm 120. It will be appreciated that any suitable system of temporarily securing the proximal aiming arm 130 to the nail aiming arm 120 may be used.

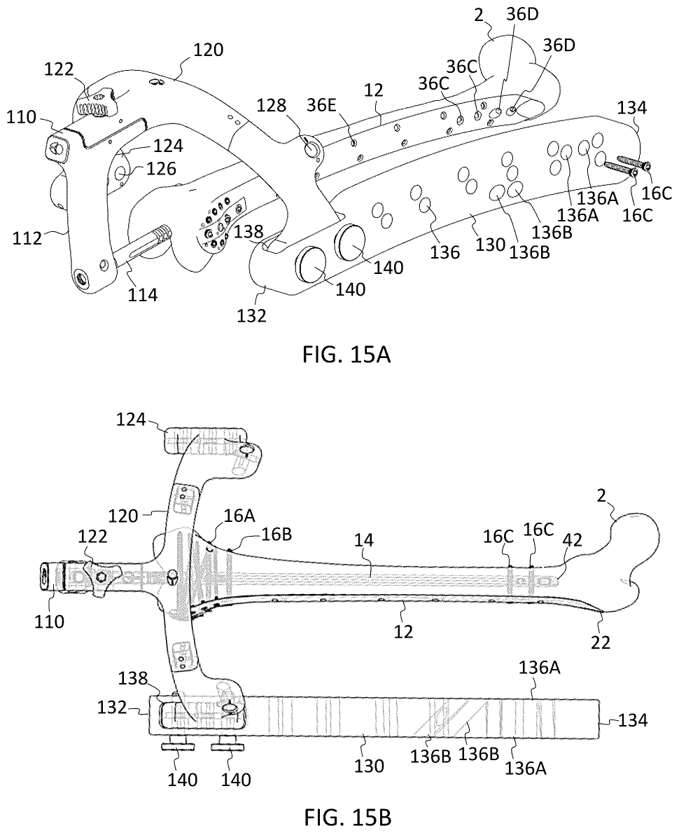

With further emphasis on FIGS. 15A-15B, a third set of screws 16 may be added to the construct 10 if desired. For example, a pair of proximal locking screws 16C may be targeted through guide openings 136A into corresponding fastener openings 36C, 44 in plate 12 and intramedullary nail 14. The proximal locking screws 16C may be targeted medially or laterally through the shaft of the femur. The interlocking screws 16C may be inserted through the proximal portion 32 of plate 12 and into the proximal end 42 of nail 14, thereby interlocking the proximal side of the plate 12 and nail 14 together. As shown in FIG. 15A, the proximal interlocking screws 16C may be aligned with the guide hole openings 136A in the proximal aiming arm 130 to enter the respective openings 36C through the proximal portion 32 of the plate 12. The proximal screws 16C may be targeted through the proximal aiming arm 130 through percutaneous incisions. The interlocking screws 16C may include screws of the same or different types, for example, as described in FIGS. 2A-5C. FIG. 15B shows the construct 10 including the proximal interlocking screws 16C through the plate 12 and nail 14. The construct stiffness may be adjusted by adding the proximal interlocking screws 16C.

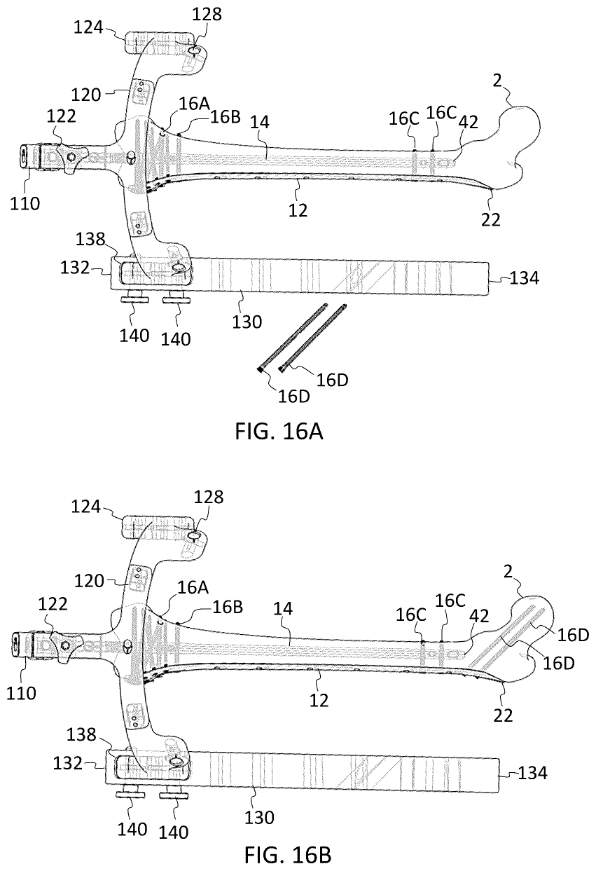

Turning now to FIGS. 16A-16B, a fourth set of prophylactic screws 16D may be inserted through the aiming arm 130 if needed. The prophylactic screws 16D may be inserted through guide openings 136B in aiming arm 130 into corresponding fastener openings 36D near the proximal end 22 of the plate 12. The prophylactic screws 16D may be angled relative to the plate 12 and proximal locking screws 16C such that the prophylactic screws 26D enter the femoral neck and head of the femur bone 2. Due to the insertion angle and location, the prophylactic screws 16D may be longer than other fasteners 16 in the construct 10. The prophylactic screws 16D pass through plate 12 but do not engage the intramedullary nail 14. In some cases, it may be useful to fortify the femoral neck and head of the femur 2 to add additional stability and facilitate healing.

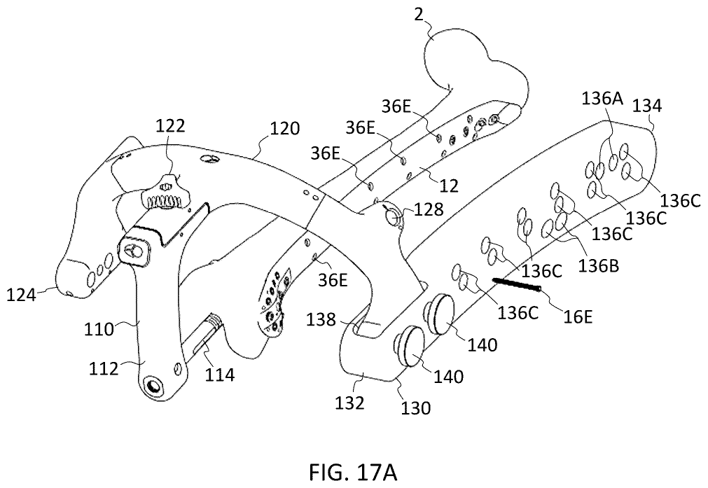

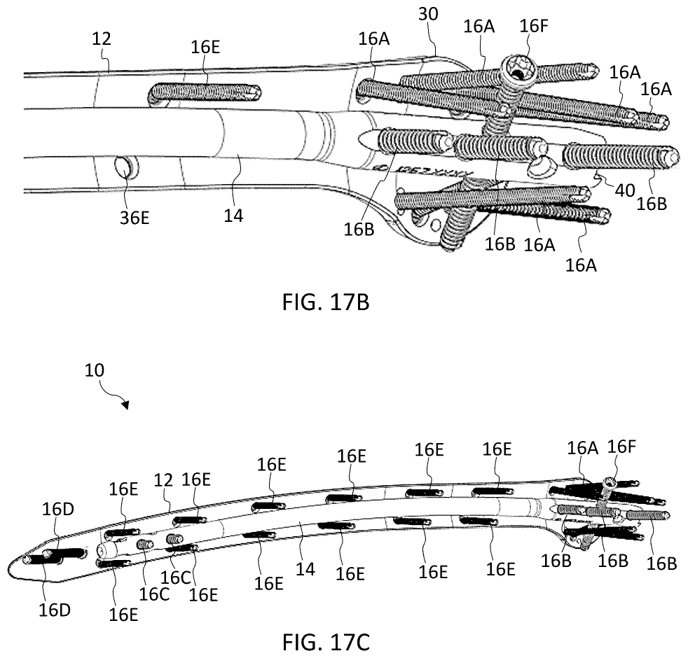

Turning now to FIGS. 17A-17C, the construct stiffness may be further modulated by adding a fifth set of locking screws 16E through the plate 12 and around the nail 14 if desired. The locking screws 16E may be inserted through guide openings 136C in the proximal aiming arm 130 and targeted into plate openings 36E and the surrounding bone 2. The proximal aiming arm 130 is configured to target holes 36E with a trajectory intended to miss the intramedullary nail 14. In other words, the screws 16E pass through plate 12 but do not engage the intramedullary nail 14. The plate locking screws 16E may be pointed medially or may be angled in any suitable direction, for example, on opposite sides of the intramedullary nail 14. In some cases, the plate locking screws 16E may be useful to fortify the femoral shaft and further modulate stiffness of the construct 10.

As shown in FIG. 17C, the locking screws 16E are inserted through plate 12 and around nail 14 with the bone omitted for clarity. One or more of a sixth type of locking screw 16F may be crossed through intramedullary nail 14 from another approach or trajectory. For example, a medial oblique locking screw 16F may be added to the distal end 40 of the intramedullary nail 14. The locking screw 16F may be targeted with nail aiming arm 120 or through another method. In one embodiment, the nail aiming arm 120 may include targeted opening 128 in each end support 124 for medial oblique access to the distal portion of the femur. Locking screw 16F passes through opening 44 in the nail 14 but does not pass through plate 12 and does not intercept any of the other fasteners 16. Screw 16F may be provided at another angle or orientation within the distal end 40 of the nail 14 to provide additional fixation in the distal femur.

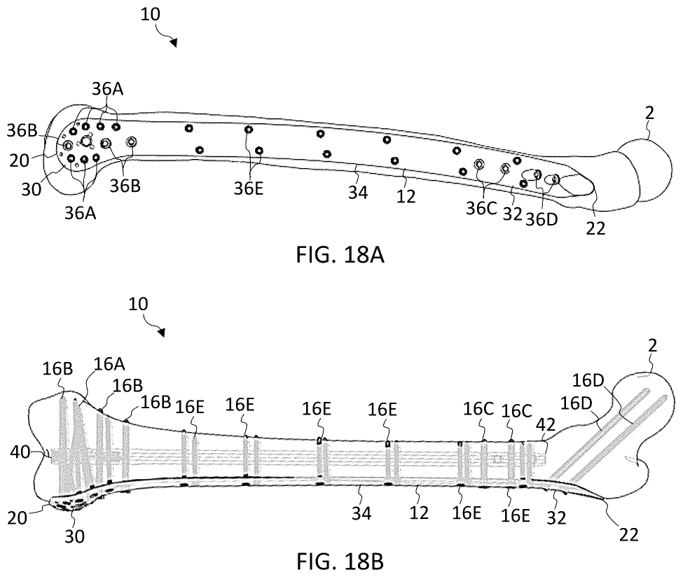

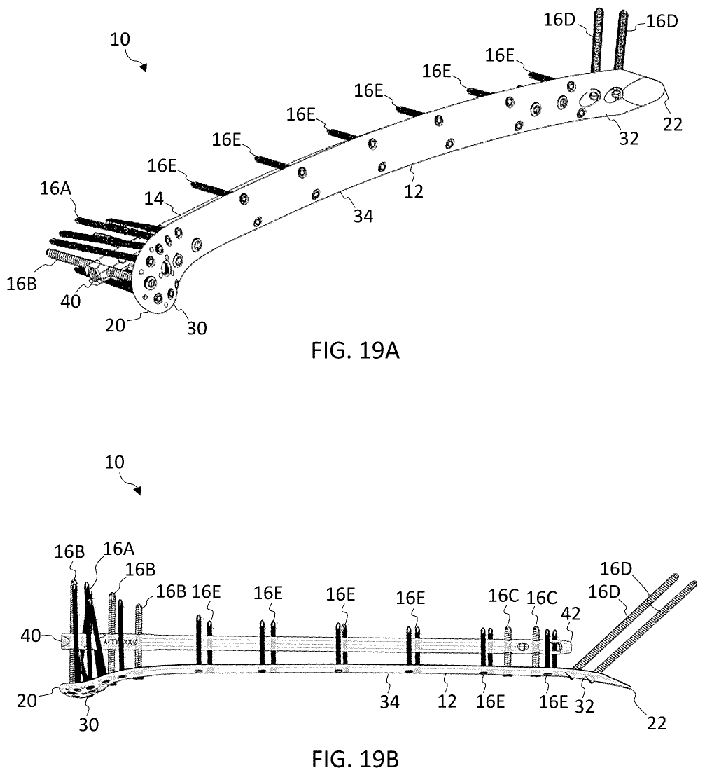

FIGS. 18A-18B show an example of a final targeted nail-plate construct 10 with the plate 12, intramedullary nail 14, and fasteners 16. FIGS. 19A-19B show the targeted nail-plate construct 10 with the bone omitted for clarity. Due to the different types and configurations of fasteners 16 including some through the plate 12 only, some interlocking the plate 12 and nail 14 together, and some through the intramedullary nail 14 only, the stiffness of the construct 10 may be precisely controlled by the surgeon and optimized for the patient. The surgeon may create a stiff construct while also allowing micro motion to optimize patient healing. The ability to choose the number and types of interlocks between the plate 12 and intramedullary nail 14 provides numerous options to control and modulate construct stiffness.

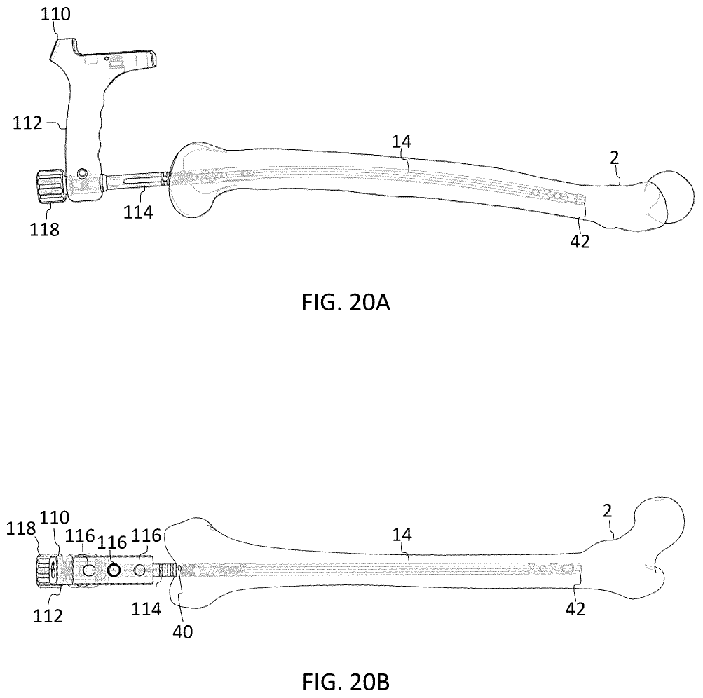

Turning now to FIGS. 20A-26, a nail first workflow for installing an interlocked targeted nail-plate construct 10 is shown according to one embodiment. Depending on the fracture type and location, a surgeon may select a nail first approach whereby the nail 14 is installed first, a fastener 16 is secured to the nail 14 and into the bone, the plate 12 is subsequently installed, and additional fasteners 16 are added as needed. The fasteners 16 may interconnect the plate 12 and intramedullary nail together, secure the plate 12 only, and/or secure the intramedullary nail 14 only.

FIGS. 20A-20B show insertion of the intramedullary nail 14 into the medullary canal of bone 2. The fracture may be reduced under fluoroscopy or through an open incision. The bone fragments may be positioned to restore length, alignment, and rotation. The nail 14 may be inserted through a standard entry portal after reducing the fracture. In this embodiment, the nail 14 is configured to be positioned through the medullary canal of a femur bone 2. The nail 14 may be installed with insertion handle 110 through a small distal incision. The coupling portion 114 of the insertion handle 110 may be temporarily secured to the distal end 40 of the nail 14 with a threaded bolt 118, for example. It will be appreciated that any suitable coupling mechanism may be employed. Although a specific type of nail and placement is shown in this embodiment, it will be appreciated that the type of nail and placement may be selected based on the surgeon's preference to treat the fracture type and location of a specific patient.

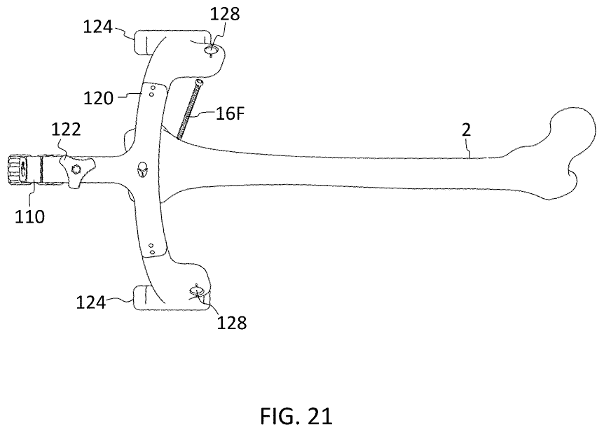

Turning now to FIG. 21, a locking fastener 16F may be inserted into the nail 14 using aiming arm 120. Aiming arm 120 may be attached to insertion handle 110 via thumb screw 122. The aiming arm 120 includes guide opening 128 in each end support 124 configured to target a medial oblique approach. The respective targeted openings 128 may provide for a medial oblique access to the distal portion of the femur and intramedullary nail 14. Once inserted, the medial oblique fastener 16F passes through opening 44 in the intramedullary nail 14 to fixate the nail 14 in the distal femur.

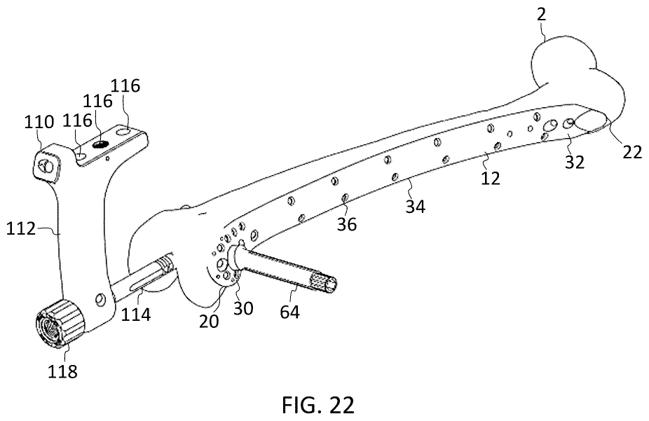

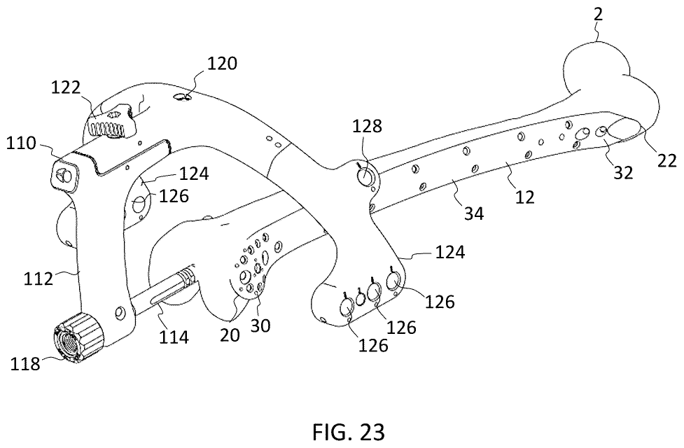

Turning now to FIG. 22, after the intramedullary nail 14 is secured, bone plate 12 is placed adjacent to an outer surface of the bone 2. The aiming arm 120 may be removed prior to placement, if necessary, to provide access. In the case of a femur plate, the plate 12 may be inserted through a small distal incision and positioned against a lateral aspect of the femur bone 2. The plate 12 may be positioned with connection shaft 64 to help facilitate insertion. When the plate 12 is positioned against the femur bone 2, the distal end 20 of the plate 12 may be placed against the lateral epicondyle of the femur and the proximal end 22 of the plate 12 may be placed toward the headed end of the femur. The plate 12 may be provisionally held in position using k-wires (not shown). Turning now to FIG. 23, the aiming arm 120 may be re-attached to the insertion handle 110. First, the connection shaft 64 may be removed and then the nail aiming arm 120 may be assembled to the insertion handle 110.

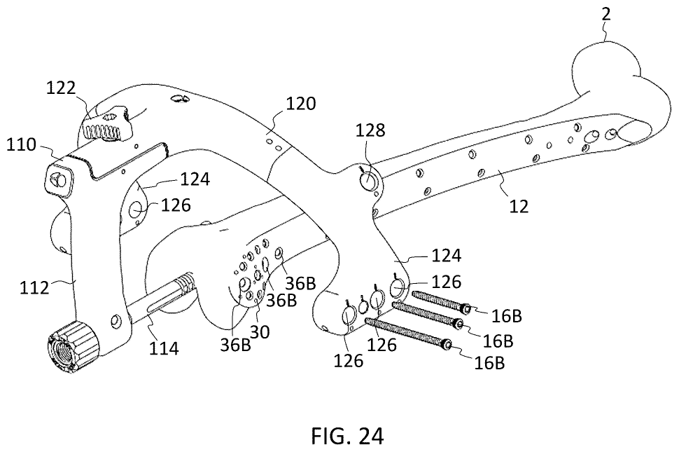

As shown in FIG. 24, depending on fracture type, location, and stiffness needed, up to three interlocking screws 16B may be inserted to interlock plate 12 to nail 14. The targeted hole openings 126 in the nail aiming arm 120 set the respective trajectories of the locking screws 16B to interface with the distal openings 36B through the plate 12 and distal openings 44 through the intramedullary nail 14. The interlocking screws 16B may thread into the intramedullary nail 12 and plate 14, may thread only into the plate 12 and pass through the nail holes 44 with clearance, or may thread into the intramedullary nail 14 and lag the plate 12 to bone 2. The interlocking screws 16B may include one or more screw types, for example, as described in FIGS. 2A-5C. The quantity and type of interlocking screws 16B affect the distal construct stiffness.

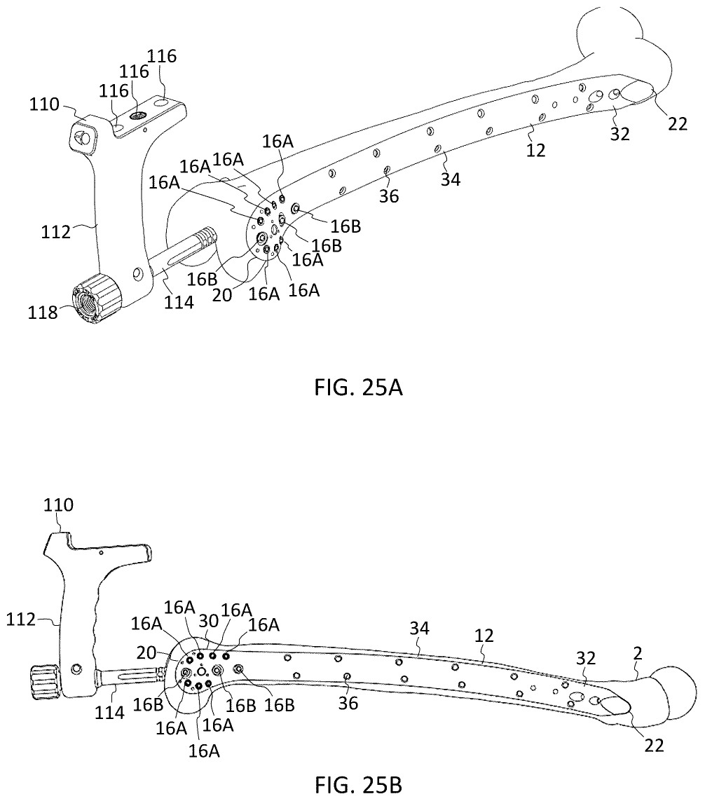

Turning now to FIGS. 25A-25B, additional distal fasteners 16A may be added as needed. After the nail aiming arm 120 is removed, the distal plate locking screws 16A may be positioned through fastener openings 36B in the distal portion 30 of the femur plate 12. The distal plate locking screws 16A may have a threaded head 46 (as shown in FIG. 2C) to lock screw 16A to the plate 12. The distal plate locking screws 16A may be nominally aimed medially and/or toward the condyles or in any suitable orientation determined by the surgeon. The distal plate locking screws 16A engage plate 12 but do not intercept nail 14. The quantity and type of distal plate locking screws 16A influence the final construct stiffness.

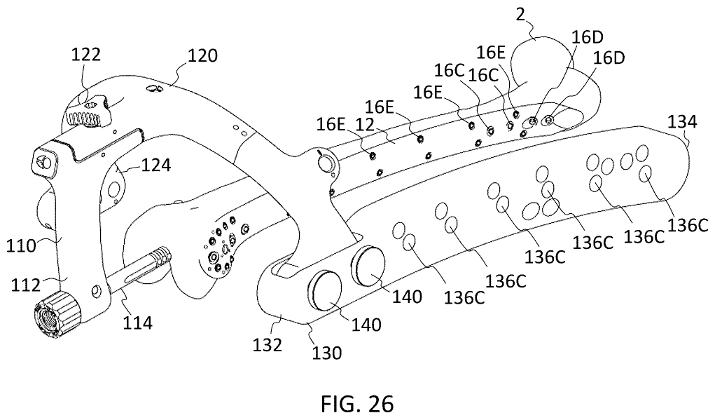

Next, the nail aiming arm 120 may be re-attached to the insertion handle 110 and the proximal aiming arm 130 may be attached to the nail aiming arm 120 as shown in FIG. 26. Additional plate locking screws 16E may be added to plate 12 as needed to control construct stiffness. The locking screws 16E may be aligned with the guide hole openings 136C in the proximal aiming arm 130 to enter the respective openings 36E through the plate 12. The locking screws 16E may be targeted through the proximal aiming arm 130 through percutaneous incisions. The plate locking screws 16E may secure plate 12 while avoiding intramedullary nail 14. If desired, additional proximal locking screws 16C, 16D may also be added to the construct, for example, using proximal aiming arm 130 in the manner previously described. The construct stiffness may be adjusted by adding different quantities and types of fasteners 16.

The nail first workflow results in a final targeted nail-plate construct 10 the same or similar to the plate first workflow, with only the order of operations changed in installing the components. The construct 10 may have many different types and configurations of fasteners 16 including some through the plate 12 only, some interlocking the plate 12 and intramedullary nail 14 together, and some through the intramedullary nail 14 only. In this manner, the stiffness of the overall construct 10 may be precisely controlled for a given patient. The construct 10 may be configured to be stiff while also allowing micro motion to optimize patient healing. The ability to choose the number and types of interlocks between the plate 12 and intramedullary nail 14 provides numerous options to control and modulate the construct stiffness.

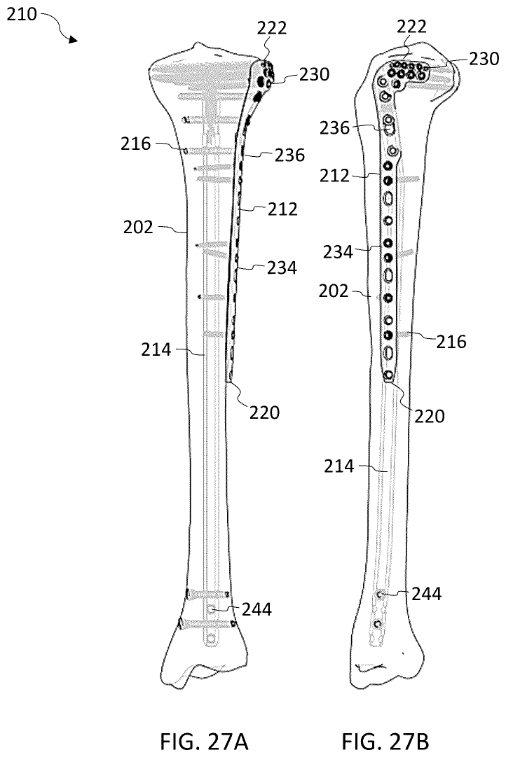

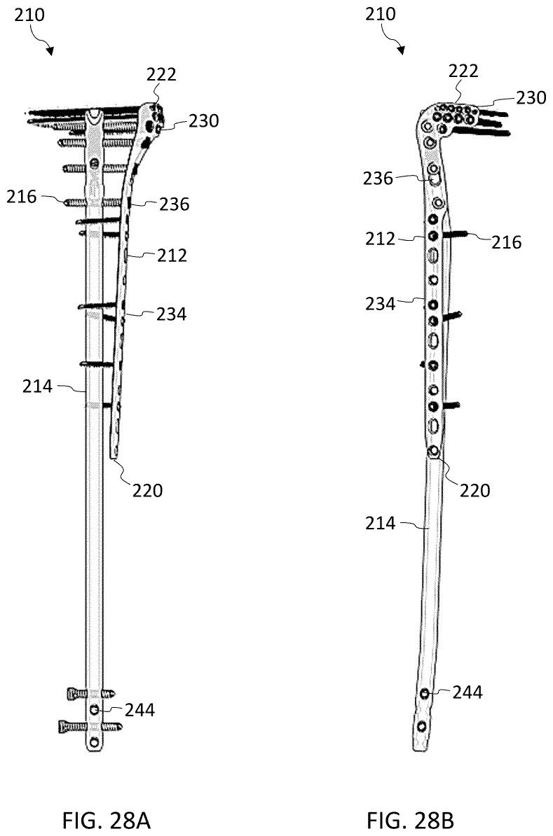

Turning now to FIGS. 27A-30B, the same plate first or nail first workflow may be applied to other anatomic areas, such as the tibia. FIGS. 27A-27B and 28A-28B illustrate the targeted plate-nail construct 210 applied to the proximal tibia 202. In this example, the proximal tibia plate 212 is applied to the lateral aspect of the tibia 202. It will be appreciated, however, that suitable tibia plates 212 may be attached to a lateral, medial or posteromedial aspect of the tibia 202 and the plates 212 are not limited to their specific locations on the tibia 202. The intramedullary nail 214 is configured to be positioned through a proximal end of the medullary canal of the tibia 202. A plurality of bone fasteners 216 are configured to pass through the plate 212 and into the tibia 202, through the plate 212 and intramedullary nail 214, and/or through the intramedullary nail 214 and into the tibia 202. When one or more fasteners 216 are interconnected between the plate 212 and intramedullary nail 214, the interlocking configuration increases the stiffness and rigidity of the overall construct 210. The construct 210 includes multiple interlocking options between the plate 212 and nail 214, which allow for customized implant constructs that are unique to the patient's needs.

The bone plate 212 comprises a distal end 220 and a proximal end 222, which are reversed relative to femur plate 12, as tibia plate 212 is oriented to the proximal end of the tibia 202. The tibia plate 212 comprises a head portion 230, for example, with an L-shape, configured to contact the lateral condyle of the tibia 202 and a shaft portion 234 configured to contact the shaft of the tibia 202. The bone plate 212 includes one or more through openings 236 each configured to receive a bone fastener 216 therethrough. The openings 236 may be configured for locking or non-locking fasteners 216 in the same manner as described for openings 36. The fastener openings 236 may include cylindrical openings, conical openings, elongated openings, threaded openings, textured openings, non-threaded and/or non-textured openings, and the like. In one embodiment, two rows of holes 236 may be defined through the head portion 230 to act as rafting holes for receiving rafting screws, which support the articular surface and prevent subsidence. Examples of proximal tibia plates are described in more detail in U.S. Pat. No. 10,368,928, which is hereby incorporated by reference in its entirety for all purposes.

The intramedullary nail 214 is positionable within the intramedullary canal of the tibia 202. The nail 214 may be introduced through the proximal end of the tibia 202. The nail 214 defines a plurality of through openings or holes 244 for receiving the fasteners 216 described herein. The fastener holes 244 may be threaded or textured (e.g., to receive locking fasteners 216) or non-threaded/non-textured (e.g., to receive compression fasteners 216). The nail 214 may come in various shapes, sizes, and designs tailored for specific bones and types of fractures.

A nail first or plate first workflow may be utilized depending on the fracture type and surgeon preference. In a plate first workflow, the following steps may be performed in any suitable order. After reducing the fracture, the plate 212 may be introduced through a proximal incision to the tibia. After aligning the plate 212, a series of rafting screws 216 may be positioned through the head 230 of the plate 212 while avoiding the exclusionary window or keep out zone 62 for the intramedullary nail 214. Subsequently, the intramedullary nail 214 may be inserted through the proximal tibia 202 and into the medullary canal. Additional proximal locking screws 216 may be used to interlock the plate 212 and intramedullary nail 214 together. The interlocking screws 216 may include screws of varying lengths or types, for example, as described in FIGS. 2A-5C. Additional plate only screws 216 may be positioned around the intramedullary nail 214. Nail only screws 216 may be added to the distal end of the intramedullary nail 214 to secure the nail 214 in the distal end of the tibia 202.

In a nail first workflow, the following steps may be performed in any suitable order. The intramedullary nail 214 is inserted through the proximal tibia 202 and into the medullary canal. The distal end of the intramedullary nail 214 may be secured with one or more nail only screws 216. The plate 212 may be placed against the proximal lateral tibia 202. The surgeon may select a suitable number and type of proximal locking screws 216 to interlock the plate 212 and intramedullary nail 214 together. Additional rafting screws 216 and/or locking screws 216 may be positioned through the proximal end of the plate 212 only and around the intramedullary nail 214.

The same locking screw strategy may be applied to control construct stiffness of the tibia construct 210. Similar instrumentation may be used for targeting and interlocking the plate 212 and intramedullary nail 214. The construct 210 may have many different types and configurations of fasteners 216 including some through the plate 212 only, some interlocking the plate 212 and nail 214 together, and some through the nail 214 only. In this manner, the stiffness of the overall construct 210 may be precisely controlled for a given patient.

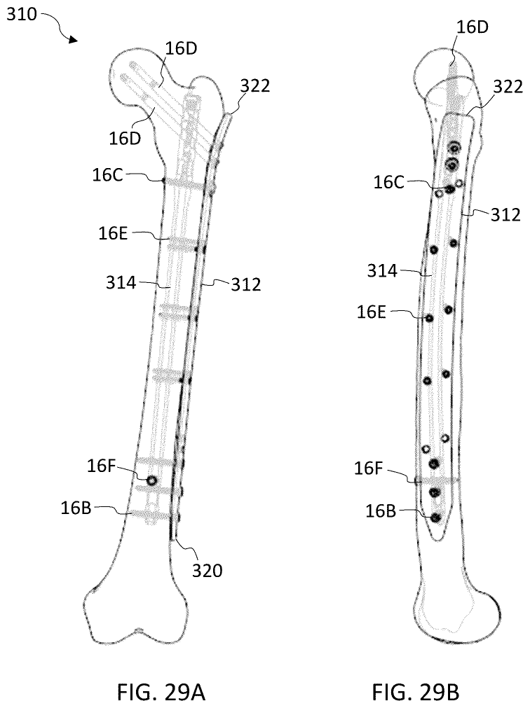

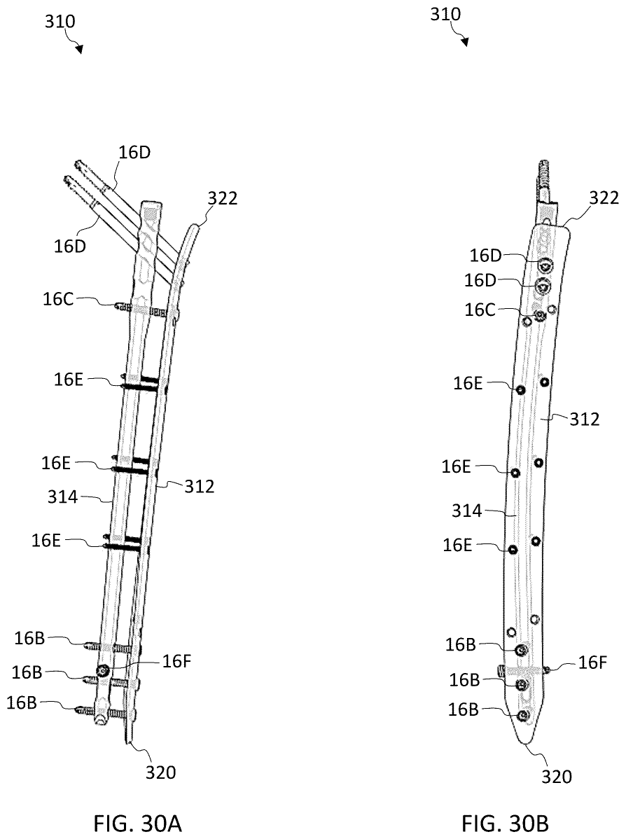

FIGS. 29A-29B and 30A-30B illustrate the targeted plate-nail construct 310 applied to the proximal femur 2. The same locking screw strategy to control construct stiffness may be applied. Similar instrumentation is used for targeting and interlocking the plate 312 and intramedullary nail 314. A nail first or plate first workflow may be utilized depending on fracture type and surgeon preference.

The bone plate 312 is reoriented for the proximal end of the femur such that the distal end 320 and proximal end 322 are reversed relative to distal femur plate 12. In this manner, the intramedullary nail 314 may be introduced through the proximal end of the femur 2. The proximal end 322 of plate 312 receives prophylactic screws 16D into the femoral neck and neck of the proximal femur. Locking screws 16C, 16B may be used to interlock the plate 312 and intramedullary nail 314 together at the proximal and distal ends, respectively. Plate locking screws 16E may be positioned around the intramedullary nail 314 and into the shaft of the femur 2. An additional nail only screw 16F may be used to secure the distal end of the intramedullary nail 314.

A nail first or plate first workflow may be utilized depending on the fracture type and surgeon preference. In a plate first workflow, the following steps may be performed in any suitable order. The proximal femur plate 312 is introduced through a proximal incision. After the plate 312 is aligned, the plate only fasteners 16E may be secured through the plate 312 while avoiding the exclusionary window or keep out zone 62 for the intramedullary nail 314. Subsequently, the intramedullary nail 314 may be inserted through the proximal femur and into the medullary canal. Prophylactic screws 16D may be used to interlock the plate 312 and intramedullary nail 314 and engage the femoral neck and head at the proximal end of the femur. Additional interlocking screws 16C may be added to the proximal end of the plate 312 and intramedullary nail 314. Up to three interlocking screws 16B may be positioned at the distal end of the plate 312 and intramedullary nail 314. The interlocking screws may include screws of varying lengths or types, for example, as described in FIGS. 2A-5C. Nail only screw 16F may be added along another trajectory to the distal end of the intramedullary nail 314 to secure the intramedullary nail 314 in the shaft toward the distal end of the femur.

In a nail first workflow, the following steps may be performed in any suitable order. The intramedullary nail 314 is inserted through the proximal femur and into the medullary canal. The distal end of the intramedullary nail 314 may be secured with one or more nail only screws 16F. The plate 312 may be placed against the proximal lateral femur. Prophylactic screws 16D may be used to interlock the plate 312 and intramedullary nail 314 and engage the femoral neck and head. Additional interlocking screw 16C may be added to the proximal end of the plate 312 and intramedullary nail 314. Up to three interlocking screws 16B may be positioned at the distal end of the plate 312 and intramedullary nail 314. Plate only fasteners 16E may be secured through the plate 312 while avoiding intramedullary nail 314. The construct 310 may have many different types and configurations of fasteners 316 including some through the plate 312 only, some interlocking the plate 312 and nail 314 together, and some through the nail 314 only. In this manner, the stiffness of the overall construct 310 may be precisely controlled.

The interlocked targeted plate-nail constructs have many advantages due to their versatility and ability to control construct stiffness according to patient needs. Targeted plate-nail constructs can address highly comminuted fractures, intra and extra-articular fractures, poor bone quality, and high body mass index (BMI) patients where plating or nailing alone would not be sufficient. The ability to plate an intra-articular fracture and also include an intramedullary nail to address a shaft fracture of the same bone allow the patient to bear weight earlier and may improve patient outcomes by controlling construct stiffness. The surgeon may create a very stiff construct at the articular surface and a construct that allows micro motion at the shaft fracture to optimize patient healing. The ability to control how many targeted interlocks are used to connect the plate to nail and what types of interlocks are used gives the surgeon unprecedented control to modulate construct stiffness. The ability to use a plate-first or nail-first workflow adds to the versatility and utility of the system. The interlocked targeted plate-nail construct may be applied to many anatomic regions using the same technique.

It will be further understood that various changes in the details, materials, and arrangements of the parts which have been described and illustrated in order to explain the nature of this invention may be made by those skilled in the art without departing from the scope of the invention as expressed in the claims. One skilled in the art will appreciate that the embodiments discussed above are non-limiting. It will also be appreciated that one or more features of one embodiment may be partially or fully incorporated into one or more other embodiments described herein.

What is claimed is:

1. A bone stabilization system comprising:
a bone plate configured to be positioned against an exterior surface of a long bone, the bone plate having a plurality of fastener openings defined therethrough;
an intramedullary nail configured to extend through a medullary canal of the long bone, the intramedullary nail defining a plurality of holes therethrough;
a first fastener configured to extend through one of the fastener openings in the bone plate and through one of the holes in the intramedullary nail and into the bone, thereby interlocking the bone plate and the intramedullary nail together;
a second fastener configured to extend through one of the fastener openings in the bone plate and directly into the bone without passing through the intramedullary nail; and
a third fastener configured to extend through one of the holes in the intramedullary nail and directly into the bone without passing through the bone plate.

2. The system of claim 1, wherein the first fastener includes a locking screw that threads into both the bone plate and the intramedullary nail, a locking screw that threads into the bone plate only, a headed or headless locking screw that threads into the nail only, or a non-locking screw that does not lock to the bone plate or the intramedullary nail.

3. The system of claim 1, wherein the second fastener includes a plate locking screw configured to secure one end of the bone plate into epiphysis of the bone.

4. The system of claim 1 further comprising a fourth fastener configured to extend through one of the fastener openings in the bone plate, around the intramedullary nail, and directly into bone without passing through the intramedullary nail.

5. The system of claim 4, wherein the fourth fastener includes a plate locking screw configured to secure the bone plate into diaphysis of the bone.

6. The system of claim 4, wherein the fourth fastener is part of a pair of fasteners positioned on opposite sides of the intramedullary nail.

7. The system of claim 1 further comprising a fifth fastener configured to extend through one of the fastener openings in the bone plate and into the neck and head of the long bone.

8. The system of claim 1, wherein the first fastener is part of one set of fasteners configured for interlocking a distal end of the bone plate and the intramedullary nail and another set of fasteners configured for interlocking a proximal end of the bone plate and the intramedullary nail.

9. A targeted plate-nail system comprising:
an intramedullary nail defining a plurality of holes therethrough;
a nail insertion handle configured for inserting the intramedullary nail into a medullary canal of a long bone, the nail insertion handle including a handle grip and a coupling portion configured to secure one end of the intramedullary nail;
a bone plate having a plurality of fastener openings defined therethrough;
a plurality of fasteners configured to extend through the fastener openings in the bone plate, through the holes in the intramedullary nail, or through both; and
a nail aiming arm attachable to the nail insertion handle to guide the plurality of fasteners into the bone plate, the intramedullary nail, or through both thereby interlocking the bone plate and the intramedullary nail together.

10. The system of claim 9 further comprising a connection shaft attachable to the bone plate.

11. The system of claim 10, wherein the connection shaft includes one or more datum pins receivable in corresponding datum recesses defined in a top face of the bone plate, and a threaded connection bolt receivable in a threaded opening in the bone plate to thereby secure the connection shaft to the bone plate.

12. The system of claim 10 further comprising a drill aiming arm and a drill sleeve attachable to the connection shaft to create an intramedullary nail entry portal.

13. The system of claim 9, wherein the nail aiming arm includes an arcuate body with opposed end supports for left and right access, each end support defines one or more targeted hole openings which align with respective fastener openings in the bone plate and holes in the intramedullary nail.

14. The system of claim 13 further comprising a proximal aiming arm attachable to the nail aiming arm with one or more connection pins.

15. The system of claim 14, wherein the proximal aiming arm defines a pocket sized and dimensioned to receive a free end of the end support of the aiming arm, thereby covering the targeted hole openings in the nail aiming arm.

16. The system of claim 14, wherein the proximal aiming arm defines a long free arm configured to align with the bone plate, the proximal aiming arm defines a plurality of guide hole openings configured to guide the plurality of fasteners into the bone plate only or the bone plate and the intramedullary nail.

17. A targeted interlocking plate-nail system comprising:
a bone stabilization implant comprising an intramedullary nail configured to extend through a medullary canal of a long bone, a bone plate configured to be positioned against an exterior surface of the long bone, and a plurality of fasteners configured to extend through the bone plate, through the intramedullary nail, or through both the bone plate and the intramedullary nail; and
a targeting instrument comprising a nail insertion handle, a nail aiming arm, and a proximal aiming arm, wherein the nail insertion handle is configured for inserting the intramedullary nail into the bone, the nail aiming arm is configured to guide some of the fasteners into the bone plate and the intramedullary nail, thereby interlocking the bone plate and the intramedullary nail together, and the proximal aiming arm is configured to guide some of the fasteners through the bone plate and the intramedullary nail or into the bone plate only.

18. The system of claim 17, wherein the plurality of fasteners include a locking screw that threads into both the bone plate and the intramedullary nail, a locking screw that threads into the bone plate only, a headed or headless locking screw that threads into the intramedullary nail only, or a non-locking screw that does not lock to the bone plate or the intramedullary nail.

19. The system of claim 17, wherein a stiffness of the bone stabilization implant is modulated by varying a number, type, and location of fasteners.

20. The system of claim 17, wherein the bone stabilization implant is installed using a plate first or nail first approach depending on the fracture type and location.
